(19) United States
(12) Patent Application Publication
(10) Pub. No.: US 2009/0292323 A1
(43) Pub. Date: Nov. 26, 2009

(54) SYSTEMS, DEVICES AND METHODS FOR POSTERIOR LUMBAR INTERBODY FUSION

(76) Inventors: Paul E. Chirico, Campbell, CA (US); Benny M. Chan, Fremont, CA (US)

Correspondence Address: SHAY GLENN LLP, 2755 CAMPUS DRIVE, SUITE 210, SAN MATEO, CA, 94403, US

(21) Appl. No.: 12/469,461

(22) Filed: May 20, 2009

Related U.S. Application Data
(60) Provisional application No. 61/054,734, filed on May 20, 2008.

(51) Int. Cl. A61B 17/56; A61F 2/44

(52) U.S. Cl. 606/86 R; 623/17.16

(57) ABSTRACT

Described herein are stabilization devices, systems and methods to aid in posterior lumbar interbody fusion (PLIF) surgeries. The stabilization devices (“devices”) described herein are typically self-expanding devices that may be implanted into an intervertebral disc and packed with a bone graft or biologic or synthetic material to promote anchoring of the stabilization device and fusion of the vertebrae adjacent to the intervertebral disc.

### CROSS REFERENCE TO RELATED APPLICATIONS

This application claims priority to U.S. Patent Application Ser. No. 61/054,734, titled “SYSTEMS, DEVICES AND METHODS FOR POSTERIOR LUMBAR INTERBODY FUSION” and filed on May 20, 2008.

This application is related to U.S. patent application Ser. No. 11/468,759, titled “Implantable Devices and Methods for Treating Micro-Architecture Deterioration of Bone Tissue”, filed Aug. 30, 2006; U.S. patent application Ser. No. 12/025,537, titled “Methods and Devices for Stabilizing Bone Compatible for use with Bone Screws”, filed Feb. 4, 2008; U.S. patent application Ser. No. 12/024,938, titled “Systems, Devices and Methods for Stabilizing Bone”, filed Feb. 1, 2008; U.S. patent application Ser. No. 12/041,607, titled “Fracture Fixation System and Method”, filed Mar. 3, 2008; and U.S. patent application Ser. No. 12/044,880, titled “Systems, Methods, and Devices for Soft Tissue Attachment to Bone”, filed Mar. 7, 2008. All of these applications are incorporated herein by reference in their entirety.

### INCORPORATION BY REFERENCE

All publications and patent applications mentioned in this specification are herein incorporated by reference in their entirety to the same extent as if each individual publication or patent application was specifically and individually indicated to be incorporated by reference.

### FIELD OF THE INVENTION

Described herein are systems, devices, and methods for performing spinal surgeries. In particular, described herein are systems, devices, and methods for performing posterior lumbar interbody fusion surgeries.

### BACKGROUND OF THE INVENTION

Posterior lumbar interbody fusion (PLIF) is a surgical procedure commonly used to treat spinal problems such as intervertebral disc degeneration, herniation, and spinal instability. Typically the procedure involves accessing the spine from the posterior, removing an intervertebral or spinal disc, and injecting a bone graft between the two vertebrae where the intervertebral disc was removed. The bone graft can stimulate the adjacent vertebrae to fuse or grow together to create an immovable section of the spine. Spinal fusion cages or other implants can be used to help hold the bone graft in place between the adjacent vertebrae to be fused.

PLIF procedures typically require a long incision along the back to access the problem portion of the spine. The length of this incision can range from approximately 1 inch to 6 inches in length. In some procedures, the paraspinous tissues surrounding the spine may need to be retracted or removed to access the intervertebral disc and adjacent vertebrae. Larger incisions and extensive tissue retraction are typically accompanied by a longer patient recovery time with more pain and discomfort. Minimally invasive approaches with smaller incisions and less tissue retraction are also being developed.

Thus, substantial challenges remain in performing PLIF surgeries. In particular, methods of performing the surgery in a minimally invasive manner would be beneficial. Described herein are devices, systems and methods that may address these and other challenges.

### SUMMARY OF THE INVENTION

Described herein are stabilization devices that may be used to stabilize tissue, or provide anchors for other implants, and method and systems including these stabilization devices systems, device and methods for using them. In particular, described herein are devices and methods for fusing adjacent vertebra.

For example, a method for fusing a patient's adjacent vertebrae may include the steps of: accessing a posterior portion of an intervertebral disc; forming a channel in the intervertebral disc; inserting an elongate stabilization device into the channel, the stabilization device having a plurality of self-expanding struts that are held in a collapsed configuration during insertion by applying force to push apart the proximal and distal ends of the stabilization device; expanding the stabilization device until the struts contact first and second vertebrae adjacent to the intervertebral disc; and injecting a flowable material around the stabilization device to promote fusion of the first and second vertebrae.

In any of the methods described herein, a channel may be formed through the intervertebral disk by first making an incision in a patient, including relatively narrow incisions. The incisions may be proximal (e.g., through the patient's back). For example, the step of accessing the posterior portion of an intervertebral disk may include making an incision into a patient that is less than 1 cm in length. From this narrow posterior opening, the disk may be accessed using any of the elongate devices (including the applicators, implants, drills, cannula, etc.) described below.

The channel may be formed through the intervertebral disk by drilling or otherwise. Insertion of the implant into the channel formed may be done through a cannula, or without a cannula. Insertion may be performed using an applicator (e.g., stabilization device applicator or inserter) that is typically hand-held, and includes an implant that is held in the collapsed (delivery) configuration at the distal end. The applicator typically includes one or more controls for releasing the force applied to hold the implant in the collapsed configuration. For example, the implant may have a first portion that is secured to the proximal end region of the elongate implant, and a second portion that is slideable or moveable with respect to the first portion and secures to the distal end region of the elongate implant. Thus the first and second portions may be slide relative to each other (and locked or held in position) to expand/collapse the implant. In some variations, the applicator may include a channel or port for the delivery of filling material, as described below.

The step of expanding may include reducing or releasing the force applied to push apart the proximal and distal ends of the stabilization device. Thus, the implant may be pre-biased in an expanded state in which the struts (which may be attached at the proximal and distal end regions of the device) extend outward from the long axis of the device.

The stabilization device may be expanded within the body (e.g., within the disc and/or vertebra) so that the distal end is stable and does not move (e.g., to withdraw or advance laterally) as the device is allowed to expand. For example, the step of expanding may include holding the distal end of the stabilization device substantially fixed and allowing the proximal end of the stabilization device to foreshorten during expansion. Controlling the expansion in this manner may prevent the device from shifting during insertion, allowing predictable placement, and may also prevent damage to tissue.

In some variations force is applied to further expand the stabilization device within the intervertebral disc. For example, additional force may be applied to draw the proximal and distal ends of the implant (using the applicator) together to further expand the implant. In some variations the implant may be removed or repositioned by reapplying force to collapse the implant after it has been completely or partially released and allowed to expand.

Each end of the implant may be released or disconnected from the applicator (e.g., the first and second portions of the applicator) after implantation. For example, the proximal and distal ends of the implant may be coupled to first and second portions of the applicator by threading; the proximal and distal ends may be counter-threaded so that the proximal end is unscrewed by rotating in a first direction (e.g., clockwise), while the distal end is unscrewed by rotating in a second direction (e.g., counterclockwise).

The stabilization device may be configured to cut through the intervertebral disc during the expanding step. Similarly, if the implant is inserted into bone (as described in some variations, below) such as a vertebra, the implant may be configured to cut the tissue, including cancellous bone, without substantially compressing it. For example, at least some of the self-expanding struts may include a cutting surface adapted to cut through the intervertebral disc.

In some variations, the method further includes the step of visualizing the stabilization device within the intervertebral disc. Thus, the implant may be marked or configured for visualization (e.g., using fluoroscope, etc.). The orientation of the implant may be controlled during implantation. For example, the implant may be held by the applicator so that the proximal end of the applicator (which remains outside of the patient, and can be manipulated by the surgeon) indicates the orientation of the struts relative to the elongate body.

In some variations, the stabilization device is formed of a shape memory material. For example, shape memory materials may include shape memory alloys (e.g., Nitinol), plastics, or the like. As mentioned, these shape memory materials may be pre-set to an expanded configuration.

The flowable material applied may be a biologic or synthetic material to promote anchoring and to allow for new bone in growth. The flowable material may be a settable material, such as a cement (e.g., PMMA), or the like. Other examples of flowable material are described below.

Also described herein are methods fusing adjacent vertebrae including the steps of accessing a posterior portion of a spine; forming a first channel in an intervertebral disc, a second channel in a first vertebra adjacent to the intervertebral disc, and a third channel in a second vertebra adjacent to the intervertebral disc; inserting a first stabilization device into the first channel, inserting a second stabilization device into the second channel, inserting a third stabilization device into the third channel, each of the stabilization devices having a plurality of self-expanding struts extendable therefrom, the struts held in an insertion configuration by applying force to push apart the proximal and distal ends of each stabilization device; expanding the stabilization devices, wherein the first stabilization device is expanded until the struts contact the first and second vertebrae; injecting a flowable material around the stabilization devices; and attaching the second stabilization device to the third stabilization device.

As mentioned above, the methods described herein may include the step of applying force to further expand the stabilization device (e.g., within the intervertebral disc and/or the vertebra). The stabilization devices may be configured to cut through the intervertebral disc and/or vertebra (e.g. cancellous bone or in some variations cortical bone) during the expanding step. As mentioned above, at least some of the self-expanding struts of the stabilization devices may include a cutting surface adapted to cut through the intervertebral disc and/or bone.

Also as mentioned above, the method may include the step of visualizing the device(s) during placement and/or expansion (e.g., within the intervertebral disc and/or vertebra), as well as when filling with flowable material and/or fixing two or more of the implants together.

Also described herein are a variety of different stabilization implants. These implants may be used in any of the methods described herein in whole or in part (e.g., to restore the height to a single vertebra, for example). In general, these stabilization implants include an elongate body having a plurality of struts configured to self-expand therefrom. The elongate body typically includes a distal and a proximal end, each of which is configured to releasably secure to a different portion of an insertion device (which may also be referred to as an applicator). The distal ends may be threaded, notched, or may otherwise include a releasable. For example, the proximal and distal ends may be threaded (in opposite directions) for attachment to different portions of an applicator so that force (e.g., tension) can be applied across the elongate length of the struts to hold them in a collapsed configuration. Examples of inserter and applicators are described in more detail in some of the patent applications incorporated by reference above.

The stabilization implants described herein may include implants having non-uniform struts. For example, the implant may have struts that have different shapes (in either the insertion/delivery or expanded configurations, or both) from other struts. In some variations the struts may have different cross-sectional shapes than other struts on the same implant. In some variations, the struts are different thicknesses or different cutting edges than other struts on the same. In addition, the different struts may be arranged to apply more or less cutting or support from different portions of the device.

For example, in some variations, one or more struts on the implant are pre-based to have an expanded shape that is more abruptly curved than other struts on the same implant. Typically, the delivery configuration for each strut is relatively flat (e.g., parallel to the long axis of the implant), so that it can be delivered in a collapsed, elongate configuration. The struts are therefore pre-biased to an expanded (or mostly expanded) shape that extends from the elongate axis of the stabilization implant. The curvature of the expanded strut shape may help contribute to the ability of the struts to cut through the tissue or to support tissue (e.g., bone). The more extreme the curve of the strut, the more readily the strut may cut the tissue. Alternatively, the surface of the strut and/or the cross-sectional shape may be configured to more readily cut (e.g., sharper and/or thinner outward-facing surfaces) may more readily cut tissue, including bone, than flatter or thicker outward-facing strut surfaces).

The arrangement of different struts around the circumference of the stabilization implant may include symmetrical arrangements (e.g., alternating configurations of struts) or arrangements including groups of more supporting or more cutting struts clustered together. The implant may also be marked (e.g., for visualization under fluoroscopy) or keyed so that the orientation of the implant, and thus the struts on the implant, can be determined before and during insertion. The inserter may include markings or other indicators (visual, tactile, etc.) indicating the location of the struts in the expanded configuration. The implants may be configured so that the struts extend outward at an angle from surface of the elongate length that is non-perpendicular. Thus, the implant may be configured so that angel between different struts extending from the implant in the expanded configuration are not equal (e.g, certain adjunct struts are closer to each other than other adjacent struts).

### BRIEF DESCRIPTION OF THE DRAWINGS

FIG. 1 shows a superior view of a normal human lumbar vertebra.

FIGS. 2A-2E are variations of stabilization devices.

FIGS. 3A and 3B are enlarged side and side perspective views (respectively) of the stabilization device shown in FIG. 2A.

FIGS. 4A and 4B are enlarged side and side perspective views (respectively) of the stabilization device shown in FIG. 2C.

FIGS. 5A and 5B are enlarged side and side perspective views (respectively) of the stabilization device shown in FIG. 2E.

FIG. 6A is one variation of a stabilization device having a plurality of continuous curvature of bending struts removably attached to an inserter.

FIG. 6B is another variation of a stabilization device removably attached to an inserter.

FIG. 7A is another variation of a stabilization device connected to an inserter. FIGS. 7B and 7C show detail of the distal and proximal ends (respectively) of the stabilization device and inserter of FIG. 7A.

FIG. 8A is one variation of a handle that may be used with an inserter.

FIGS. 8B-8E illustrate connecting an inserter to a handle such as the handle of FIG. 8A.

FIGS. 9A-9D illustrate the operation of an inserter and handle in converting a stabilization device from a relaxed, deployed configuration (in FIGS. 9A and 9B) to a contracted, delivery configuration (in FIGS. 9C and 9D).

FIG. 10 is one variation of an inserter connected to a stabilization device within an access cannula.

FIG. 11 shows one variation of a trocar and access cannula.

FIG. 12A-12C shows one variation of a hand drill.

FIG. 13 shows one variation of a cement cannula and two cement filling devices.

FIGS. 14A-14D show different variations of an access cannula that may be used with a stabilization device and inserter, trocar, drill, and cement cannula, respectively.

FIGS. 15A-15G illustrate one method of treating a bone.

FIGS. 16A-16B illustrate one method of using bone cement with the stabilization devices described herein.

FIGS. 17A-17D show perspective (17A and 17B) and cross-sectional (17C and 17D) views of one variation of a stabilization device that may be used in a posterior interbody lumbar fusion (PLIF) surgery.

FIGS. 18A-18D show perspective (18A and 18B) and cross-sectional (18C and 18D) views of one variation of a stabilization device that may be used in a PLIF surgery.

FIGS. 18E-18G show cross-sectional views through alternative variations of stabilization devices as described herein.

FIGS. 19A-19D show various configurations of struts that may be used to form a portion of a stabilization device as described herein.

FIG. 19E shows one variation of a stabilization device having a non-uniform strut shape.

FIGS. 20A-20D show perspective (20A and 20B) and cross-sectional (20C and 20D) views of one variation of a stabilization device that may be used in a PLIF surgery.

FIG. 21 illustrates a section of a human spine including intervertebral disc and two adjacent vertebrae.

FIGS. 22A-22E illustrate one method of performing a PLIF surgery utilizing a stabilization device, such as the stabilization devices described in FIGS. 17A-20D.

FIGS. 23A-23F illustrate one method of performing a PLIF surgery utilizing multiple stabilization devices, such as the stabilization devices described in FIGS. 17A-20D.

FIG. 23G illustrates an alternative step for the method shown in FIGS. 23A-23E.

### DETAILED DESCRIPTION OF THE INVENTION

The stabilization devices, systems and methods described herein may aid in posterior lumbar interbody fusion (PLIF) surgeries. The stabilization devices (also referred to as simply “devices”) described herein may be implanted into an intervertebral disc and packed with a bone graft or biologic or synthetic material to promote anchoring of the stabilization device and fusion of the vertebrae adjacent to the intervertebral disc. The devices, systems and methods described herein may be used in any appropriate body region, particularly in the lower back or lumbar region of the spine.

In general, the stabilization devices described herein include a self-expanding elongate shaft that may be positioned within an intervertebral disc and/or within a vertebra and expanded to anchor within that portion of the spine. The elongate shaft may include a plurality of self-expanding struts that expand to form a bow-shape. The stabilization device may also typically include one or more attachment regions adapted to attach other stabilization devices thereto. Examples and illustrations of different variations of these stabilization implants are provided below.

The stabilization devices described herein may self-expand from a compressed profile having a relatively narrow diameter (e.g., a delivery configuration) into an expanded profile (e.g., a deployed configuration). A stabilization device generally includes an elongate shaft forming a plurality of struts that may extend from the shaft body. The shaft is referred to as elongate since it extends linearly in the delivery configuration. The distal and proximal regions of the stabilization device, and particularly the region surrounding the elongate shaft, may include one or more attachment regions configured to attach to an inserter for inserting (and/or removing) the entire stabilization device. The inserter (also referred to as a delivery device) is generally configured to apply force to maintain the stabilization device in the delivery conformation until it has been at inserted at least partially into the bone.

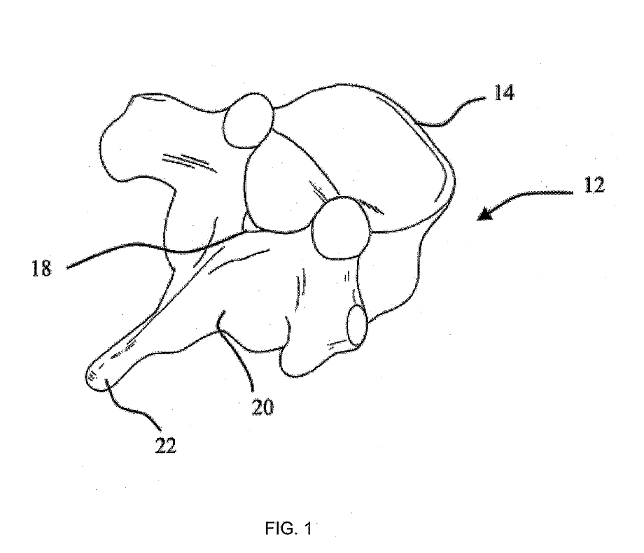

In some variations, as described herein, the stabilization devices may be placed both in the intervertebral space as well as within one or more adjacent vertebra. FIG. 1 shows a superior plan view of a normal human lumbar vertebra 12. Although human lumbar vertebrae vary somewhat according to location, the vertebrae share many common features. Each vertebra 12 includes a vertebral body 14. Two short boney protrusions, the pedicles, extend dorsally from each side of the vertebral body 14 to form a vertebral arch 18 which defines the vertebral foramen. A spinal disc (not shown) is located between adjacent vertebras (in the intervertebral space).

At the posterior end of each pedicle, the vertebral arch 18 flares out into broad plates of bone known as the laminae 20. The laminae 20 fuse with each other to form a spinous process 22. The spinous process provides for muscle and ligamentous attachment. A smooth transition from the pedicles to the laminae is interrupted by the formation of a series of processes. Two transverse processes thrust out laterally, one on each side, from the junction of the pedicle with the lamina. The transverse processes serve as levers for the attachment of muscles to the vertebrae. Four articular processes, two superior and two inferior, also rise from the junctions of the pedicles and the laminae. The superior articular processes are sharp oval plates of bone rising upward on each side of the vertebrae, while the inferior processes are oval plates of bone that jut downward on each side.

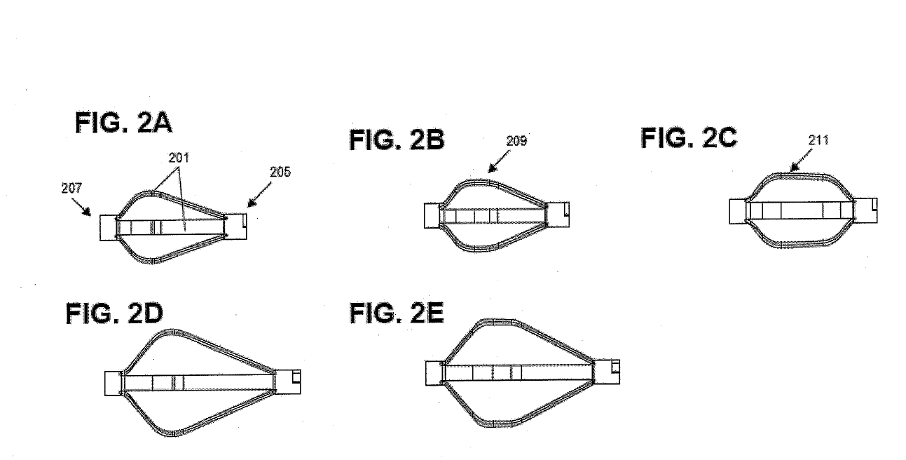

FIGS. 2A through 6 show exemplary stabilization devices. Side profile views of five variations of stabilization devices are shown in FIGS. 2A through 2E. FIG. 2A shows a 10 mm asymmetric stabilization device in an expanded configuration. The device has four struts 201, 201′, formed by cutting four slots down the length of the shaft. In this example, the elongate expandable shaft has a hollow central lumen, and a proximal end 205 and a distal end 207. By convention, the proximal end is the end closest to the person inserting the device into a subject, and the distal end is the end furthest away from the person inserting the device.

The struts 201, 201′ of the elongate shaft is the section of the shaft that projects from the axial (center) of the shaft. Three struts are visible in each of FIGS. 2A-2E. In general, each strut has a leading exterior surface that forms a cutting surface adapted to cut through cancellous bone as the strut is expanded away from the body of the elongate shaft. This cutting surface may be shaped to help cut through the cancellous bone (e.g., it may have a tapered region, or be sharp, rounded, etc.). In some variations, the cutting surface is substantially flat.

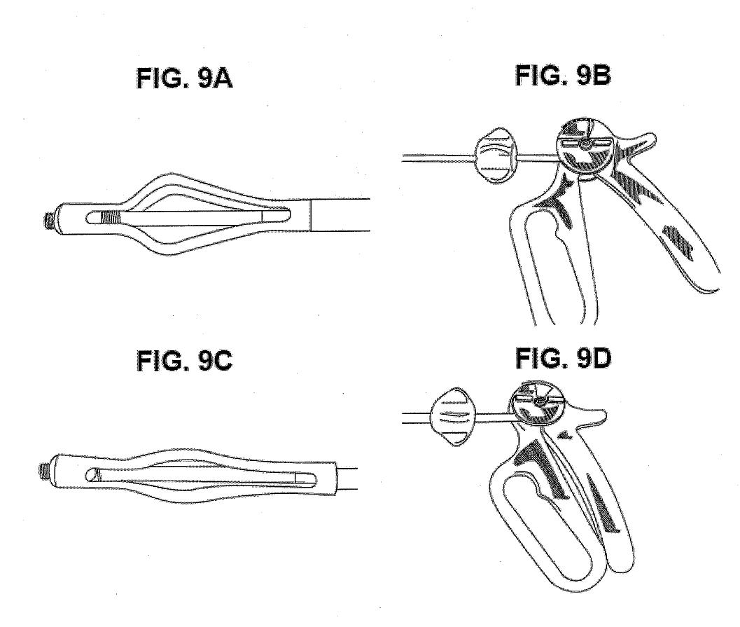

The stabilization device is typically biased so that it is relaxed in the expanded or deployed configuration, as shown in FIGS. 2A to 2E. In general, force may be applied to the stabilization device so that it assumes the narrower delivery profile, described below (and illustrated in FIG. 9C). Thus, the struts may elastically bend or flex from the extended configuration to the unextended configuration.

The struts in all of these examples are continuous curvature of bending struts. Continuous curvature of bending struts are struts that do not bend from the extended to an unextended configuration (closer to the central axis of the device shaft) at a localized point along the length of the shaft. Instead, the continuous curvature of bending struts are configured so that they translate between a delivery and a deployed configuration by bending over the length of the strut rather than by bending at a discrete portion (e.g., at a notch, hinge, channel, or the like). Bending typically occurs continuously over the length of the strut (e.g., continuously over the entire length of the strut, continuously over the majority of the length of the strut (e.g., between 100-90%, 100-80%, 100-70%, etc.), continuously over approximately half the length of the strut (e.g., between about 60-40%, approximately 50%, etc.).

The “curvature of bending” referred to by the continuous curvature of bending strut is the curvature of the change in configuration between the delivery and the deployed configuration. The actual curvature along the length of a continuous curvature of bending strut may vary (and may even have “sharp” changes in curvature). However, the change in the curvature of the strut between the delivery and the deployed configuration is continuous over a length of the strut, as described above, rather than transitioning at a hinge point. Struts that transition between delivery and deployed configurations in such a continuous manner may be stronger than hinged or notched struts, which may present a pivot point or localized region where more prone to structural failure.

Thus, the continuous curvature of bending struts do not include one or more notches or hinges along the length of the strut. Two variations of continuous curvature of bending struts are notchless struts and/or hingeless struts. In FIG. 2A, the strut 201 bends in a curve that is closer to the distal end of the device than the proximal end (making this an asymmetric device). In this example, the maximum distance between the struts along the length of device is approximately 10 mm in the relaxed (expanded) state. Thus, this may be referred to as a 10 mm asymmetric device.

FIG. 2B shows another example of a 10 mm asymmetric device in which the curve of the continuous curvature of bending strut has a more gradual bend than the devices shown in FIG. 2A. This variation may be particularly useful when the device is used to abut or support non-cancellous bone in the deployed state. For example, the flattened curved region 209 of the continuous curvature of bending strut may provide a contact surface to support or abut the non-cancellous bone. For example, the leading edge of the strut (the cutting edge) may expand through the cancellous bone and abut the harder cortical bone forming the exterior shell of the bony structure. FIG. 2C shows a symmetric 10 mm device in which this concept 211 is even more fully developed. FIGS. 2D and 2E are examples of 18 mm devices similar to the 10 mm devices shown in FIGS. 2A and 2B, respectively.

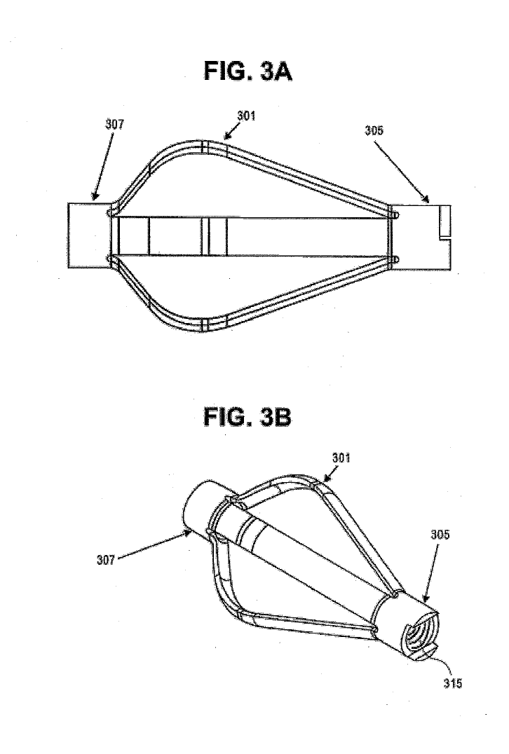

FIGS. 3A and 3B show enlarged side and side perspective views (respectively) of the mm asymmetric device shown in FIG. 2A. These figures help further illustrate the continuous curve of the continuous curvature of bending strut 301. The proximal end (the end facing to the right in FIGS. 3A and 3B), shows one variation of an attachment region to which the device may be attached to one portion of an introducer. In this example, the end includes a cut-out region 305, forming a seating area into which a complementary attachment region of an inserter may mate. Although not visible in FIGS. 3A and 3B, the distal region 307 of the device may also include an attachment region. In some variations, the inner region (and/or outer region) of the proximal end 315 of the device may be threaded. Threads may also be used to engage the inserter at the proximal (and/or distal) ends of the device as part of the attachment region.

An attachment region may be configured in any appropriate way. For example, the attachment region may be a cut-out region (or notched region), including an L-shaped cut out, an S-shaped cut out, a J-shaped cut out, or the like, into which a pin, bar, or other structure on the inserter may mate. In some variations, the attachment region is a threaded region which may mate with a pin, thread, screw or the like on the inserter. In some variations, the attachment region is a hook or latch. The attachment region may be a hole or pit, with which a pin, knob, or other structure on the inserter mates. In some variations, the attachment region includes a magnetic or electromagnetic attachment (or a magnetically permeable material), which may mate with a complementary magnetic or electromagnet region on the inserter. In each of these variations the attachment region on the device mates with an attachment region on the inserter so that the device may be removably attached to the inserter.

The stabilization devices described herein generally have two or more releasable attachment regions for attaching to an inserter. For example, a stabilization device may include at least one attachment region at the proximal end of the device and another attachment region at the distal end of the device. This may allow the inserter to apply force across the device (e.g., to pull the device from the expanded deployed configuration into the narrower delivery configuration), as well as to hold the device at the distal end of the inserter. However, the stabilization devices may also have a single attachment region (e.g., at the proximal end of the device). In this variation, the more distal end of the device may include a seating region against which a portion of the inserter can press to apply force to change the configuration of the device. In some variations of the self-expanding stabilization devices, the force to alter the configuration of the device from the delivery to the deployed configuration comes from the material of the device itself (e.g., from a shape-memory material), and thus only a single attachment region (or one or more attachment region at a single end of the device) is necessary.

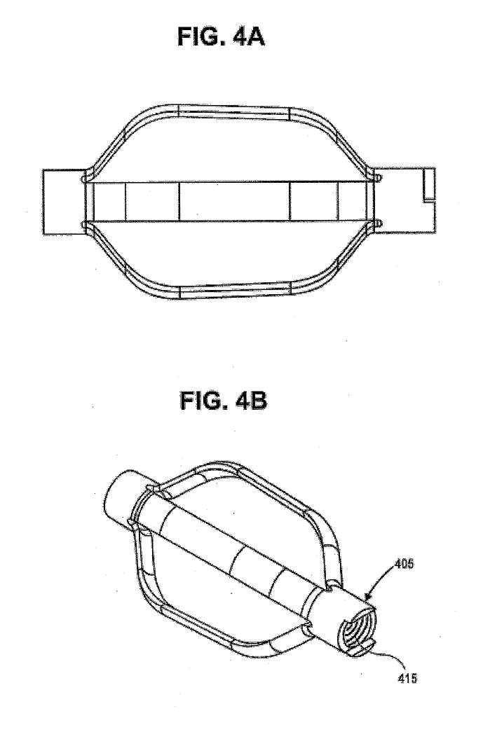

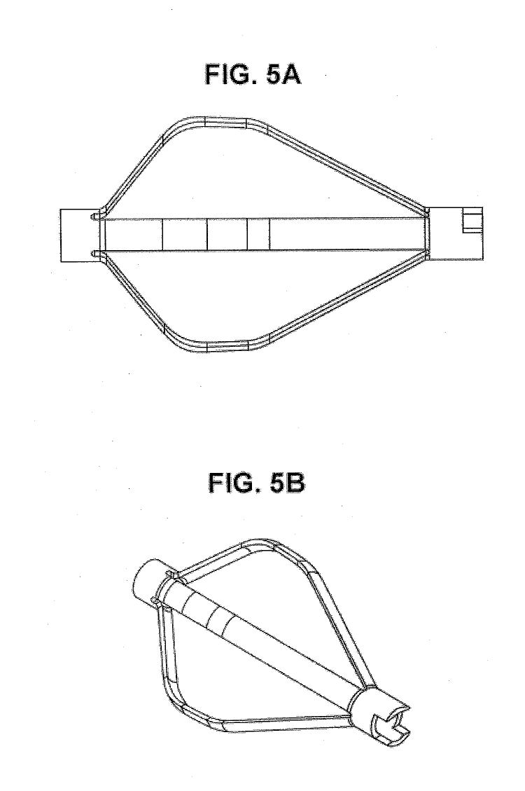

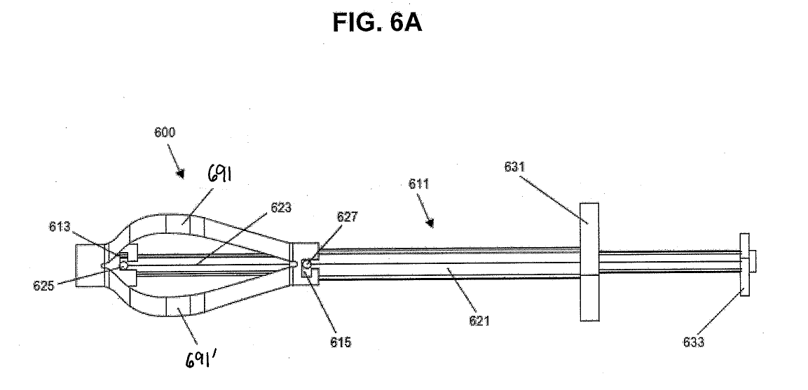

Similar to FIGS. 3A and 3B, FIGS. 4A and 4B show side and side perspective views of exemplary symmetric 10 mm devices, and FIGS. 5A and 5B show side and side perspective views of 18 mm asymmetric devices.

The continuous curvature of bending struts described herein may be any appropriate dimension (e.g., thickness, length, width), and may have a uniform cross-sectional thickness along their length, or they may have a variable cross-sectional thickness along their length. For example, the region of the strut that is furthest from the tubular body of the device when deployed (e.g., the curved region 301 in FIGS. 3A and 3B) may be wider than other regions of the strut, providing an enhanced contacting surface that abuts the non-cancellous bone after deployment.

The dimensions of the struts may also be adjusted to calibrate or enhance the strength of the device, and/or the force that the device exerts to self-expand. For example, thicker struts (e.g., thicker cross-sectional area) may exert more force when self-expanding than thinner struts. This force may also be related to the material properties of the struts.

As mentioned, in some variations, different struts on the device may have different widths or thicknesses. In some variations, the same strut may have different widths of thicknesses along its length. Controlling the width and/or thickness of the strut may help control the forces applied when expanding. For example, controlling the thickness may help control cutting by the strut as it expands.

Similarly, the width of the strut (including the width of the outward-facing face of the strut) may be controlled. The outward-facing face may include a cutting element (e.g., a sharp surface) along all or part of its width, as mentioned.

Varying the width, thickness and cutting edge of the struts of a device may modulate the structural and/or cutting strength of the strut. This may help vary or control the direction of cutting. Another way to control the direction of cutting is to modify the pre-biased shape. For example, the expanded (pre-set) shape of the struts may include one or more struts having a different shape than the other struts. For example, one strut may be configured to expand less than the other struts, or more than other struts. Thus, in some variations, the shape of the expanded implant may have an asymmetric shape, in which different struts have different expanded configurations.

The struts may be made of any appropriate material. In some variations, the struts and other body regions are made of substantially the same material. Different portions of the stabilization device (including the struts) may be made of different materials. In some variations, the struts may be made of different materials (e.g., they may be formed of layers, and/or of adjacent regions of different materials, have different material properties). The struts may be formed of a biocompatible material or materials. It may be beneficial to form struts of a material having a sufficient spring constant so that the device may be elastically deformed from the deployed configuration into the delivery configuration, allowing the device to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol), tantalum, tantalum alloys, aluminum, etc. Any appropriate shape memory material, including shape memory alloys such as Nitinol may also be used.

Other regions of the stabilization device may be made of the same material(s) as the struts, or they may be made of a different material. Any appropriate material (preferably a biocompatible material) may be used (including any of those materials previously mentioned), such as metals, plastics, ceramics, or combinations thereof. In variations where the devices have bearing surfaces (i.e. surfaces that contact another surface), the surfaces may be reinforced. For example, the surfaces may include a biocompatible metal. Ceramics may include pyrolytic carbon, and other suitable biocompatible materials known in the art. Portions of the device can also be formed from suitable polymers include polyesters, aromatic esters such as polyalkylene terephthalates, polyamides, polyalkenes, poly(vinyl) fluoride, PTFE, polyarylethyl ketone, and other materials. Various alternative embodiments of the devices and/or components could comprise a flexible polymer section (such as a biocompatible polymer) that is rigidly or semi rigidly fixed.

The devices (including the struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the stabilization devices may elute a bioactive substance to promote or inhibit bone growth, vascularization, etc. In one variation, the device includes an elutible reservoir of bone morphogenic protein (BMP).

As previously mentioned, the stabilization devices may be formed about a central elongate hollow body. In some variations, the struts are formed by cutting a plurality of slits long the length (distal to proximal) of the elongate body. This construction may provide one method of fabricating these devices, however the stabilization devices are not limited to this construction. If formed in this fashion, the slits may be cut (e.g., by drilling, laser cutting, etc.) and the struts formed by setting the device into the deployed shape so that this configuration is the default, or relaxed, configuration in the body. For example, the struts may be formed by plastically deforming the material of the struts into the deployed configuration. In general, any of the stabilization devices may be thermally treated (e.g., annealed) so that they retain this deployed configuration when relaxed. Thermal treatment may be particularly helpful when forming a strut from a shape memory material such as Nitinol into the deployed configuration.

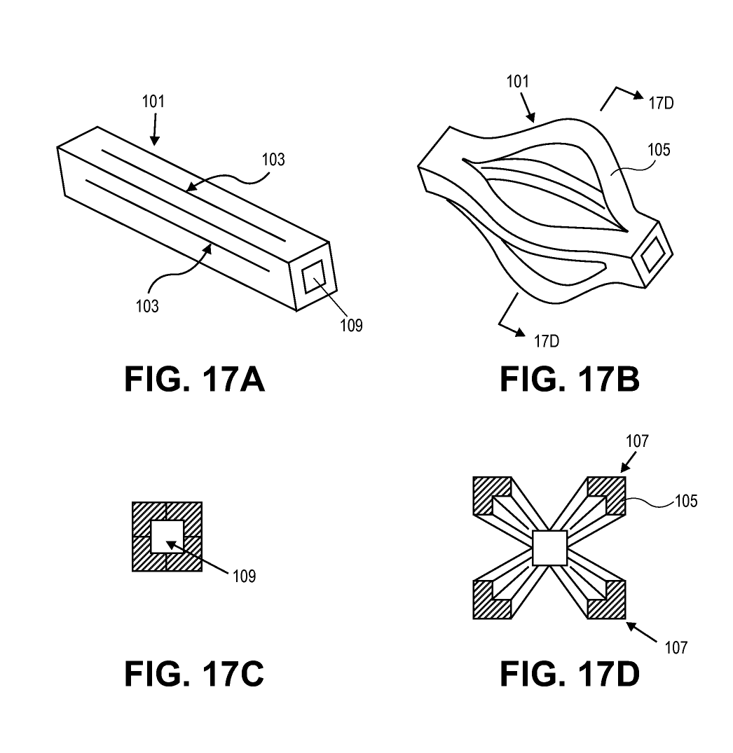

Other variations of stabilization device may also be used. For example, jumping ahead, FIGS. 17A to 17D illustrate one variation of a stabilization device 101 that forms struts with sharp cutting edges that may help the device insert into a spine and secure it in place. FIG. 17A shows a perspective view of just the elongate shaft of a stabilization device. The other features and elements of a stabilization device (e.g., attachment and connector regions) have been removed to illustrate just features of the elongate shaft. It should be understood that this example of a stabilization device may be used with any of the devices described herein including the features not illustrated in FIGS. 17A-17D.

FIG. 17A shows a stabilization device in the unexpanded or delivery configuration, and FIG. 17B shows the same stabilization device in an expanded configuration. As mentioned, the stabilization device may be pre-biased in the expanded configuration. For example, the stabilization device may be formed at least partially of a shape memory material that is configured so that the relaxed state at body temperature is the expanded state. Force may be applied to hold the device in the linear, delivery configuration, shown in FIG. 17A. For example, force may be applied radially (e.g., within a sleeve or cannula) or by applying tension (e.g., pulling on either end of the stabilization device. The stabilization device 101 of FIG. 17A is formed as a four-sided (rectangular or square) cylinder having four silts 103 cut partially along the length to form the four struts 105. As shown in FIG. 17D, the struts 105 each include a cutting edge region 107 formed by the corner of the rectangular cylinder when the struts expand. The inner diameter of the stabilization device shown is hollow 109, which may allow for the delivery of a flowable material including a cement or bone graft material, as described below.

FIGS. 17C and 17D show cross-sections through the stabilization device. For example, FIG. 17C shows a cross-section through the middle of the delivery configuration shown in FIG. 17A. FIG. 17D shows a cross section through the middle region of the expanded configuration shown in FIG. 1B, as indicated by the arrows 17D. In FIG. 17D, the cutting edge region 107 of the struts 105 is apparent. In this example, the cutting edge is approximately a 90 degree angle edge. In other variations the edge may be made sharper by increasing the curvature of the edge region.

The stabilization device embodiment shown in FIGS. 17A-17D illustrates one variation of a stabilization device including an elongate shaft forming struts 105 that may cut through bone or intervertebral disc during expansion from a collapsed delivery profile to an expanded deployed profile. Edge region 107 provides a sharp or cutting surface on the self-expanding struts 105 that allows the elongate shaft to apply a cutting force to the bone or intervertebral disc during expansion from the collapsed delivery profile to the expanded deployed profile.

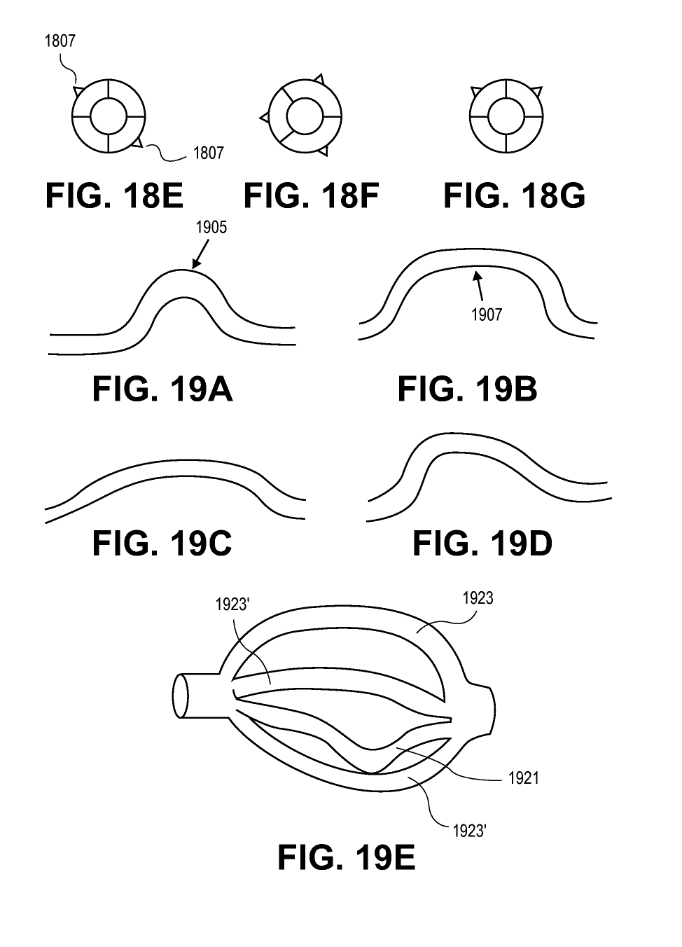

Other variations of stabilization devices may also include cutting edges. As shown in FIGS. 18A-18D, a stabilization device 1801 with an elongate shaft having a cylindrical or round cross-section (e.g., FIGS. 18C-18D) can have an additional edge or protrusion 1807 extending from the self-expanding struts to provide a cutting force to bone or intervertebral disc during expansion. Any of the variations shown in FIGS. 17A-20D may also include the releasable attachment regions at the proximal and distal end (not visible in these figures for simplicity).

FIGS. 18E-18G illustrate other variations of implants that include struts having different configurations, as mentioned above. For example, in FIG. 18E (compared to FIG. 18C, described above) is a cross-section through a middle region of an implant being held in a collapsed (e.g., delivery) configuration. In this variation the struts that are different. Two of the four struts (the opposite two) include cutting surfaces 1807, while the adjacent struts do not. In the variation shown in FIG. 18F, the device include three struts (rather than four). In FIG. 18G, two cutting struts are adjacent to each other and opposite two less-cutting (e.g., supporting) struts. In some variations, the width of the strut (e.g., the portion of the struts cut out from the cylinder in the collapsed configuration) is different between different struts. For example, struts that are more supportive than cutting may be larger (e.g., may form more of the angular radius of the collapsed cylinder), while more cutting struts may be thinner.

FIGS. 19A-19D illustrate different expanded shapes for struts that may form part of a stabilization device. For example, in FIG. 19A the strut includes a central region 1905 that is more steeply curved than other strut configurations (e.g., compare to FIGS. 19B-19D). This strut may apply a different force, and therefore a different cutting moment, than other struts, such as those shown in FIGS. 19B and 19D. In FIG. 19B, the strut has a relatively flat central region 1907 that may support tissue (e.g., bone) and be less penetrating than the configuration shown in FIG. 19A. FIG. 19C illustrates another variation similar to FIG. 19B. FIG. 19D shows a strut that is similar to the variation shown in FIGS. 3A and 3B. Any of the struts shown in FIGS. 19A-19D may be used in combination with each other or with other strut configurations, within the same device. For example, FIG. 19E illustrates one variation of a stabilization device including two different strut configurations, similar to those shown in FIGS. 19A 1921 and 19B 1923.

Different strut configurations, including difference in expanded configurations, different thicknesses, etc. may effect on the way in which the strut interacts with the tissue, both as it expands and after it is implanted over the longer term. For example, the shaper, more extremely curved struts (e.g., in FIG. 19E, strut 1921) may cut through the tissue, including the bone, more readily than the broader (more supporting) struts (e.g., struts 1923, 1923′). Thus, the implant may be inserted so that the more cutting struts face (in the delivery configuration) regions to be cut.

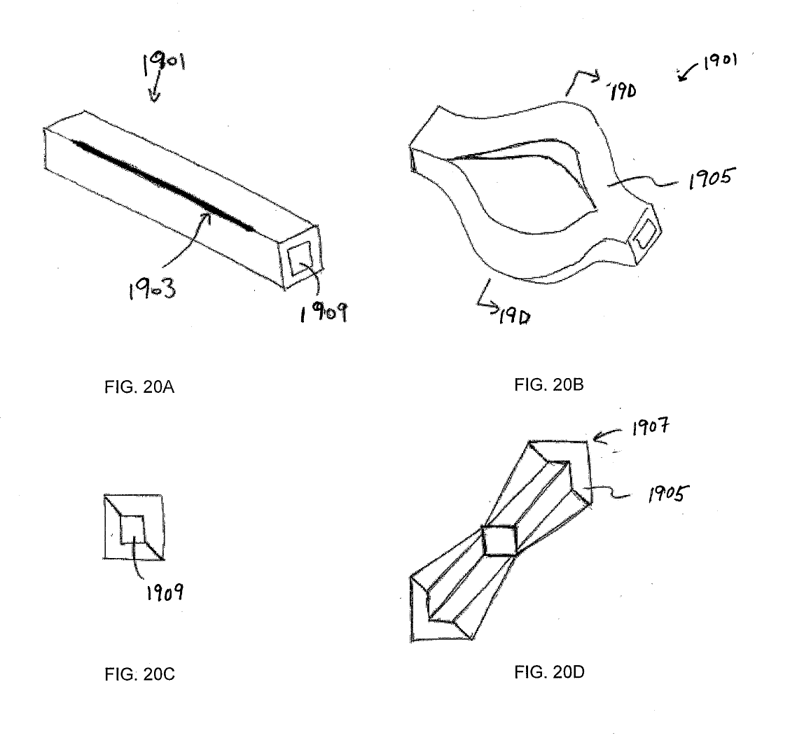

The stabilization device embodiment shown in FIGS. 20A-20D illustrates one variation of a stabilization device having two slits 1903, which expands to form two struts 1905 upon expansion from the collapsed delivery profile to the expanded deployed profile. FIGS. 20C and 20D show a cross-section through the middle of the delivery configuration shown in FIG. 20A and through the middle of the expanded configuration shown in FIG. 20B, respectively. As described above, this embodiment of the stabilization device also includes cutting edges 1907 to provide a cutting force to a vertebra or an intervertebral disc during expansion.

As mentioned, the dimensions of the struts may be adjusted to calibrate or enhance the strength of the elongate shaft, and/or the force that the elongate shaft exerts when self-expanding. For example, thicker struts (e.g., thicker cross-sectional area) may exert more force when self-expanding than thinner struts. This force may also be related to the material properties of the struts.

The struts may be made of any appropriate material. In some variations, the struts and other regions of the stabilization device are made of substantially the same material. Different portions of the stabilization device (including the struts) may be made of different materials. In some variations, the struts may be made of different materials (e.g., they may be formed of layers, and/or of adjacent regions of different materials, which may have different material properties). The struts may be formed of a biocompatible material or materials. It may be beneficial to form struts of a material having a sufficient spring constant so that the device may be elastically deformed from the deployed configuration into the delivery configuration, allowing the elongate shaft to self-expand back to approximately the same deployed configuration. In some variation, the strut is formed of a shape memory material that may be reversibly and predictably converted between the deployed and delivery configurations. Thus, a list of exemplary materials may include (but is not limited to): biocompatible metals, biocompatible polymers, polymers, and other materials known in the orthopedic arts. Biocompatible metals may include cobalt chromium steel, surgical steel, titanium, titanium alloys (such as the nickel titanium alloy Nitinol™), tantalum, tantalum alloys, aluminum, etc. Any appropriate shape memory material, including shape memory alloys such as Nitinol™ may also be used.

Other portions of the stabilization device may be made of the same material(s) as the struts, or they may be made of a different material. Any appropriate material (preferably a biocompatible material) may be used, including any of those materials previously mentioned, such as metals, plastics, ceramics, or combinations thereof. In some variations, portions of the stabilization device can also be formed from suitable polymers include polyesters, aromatic esters such as polyalkylene terephthalates, polyamides, polyalkenes, poly(vinyl)fluoride, PTFE, polyarylethyl ketone, and other materials. Various alternative embodiments of the stabilization device and/or components could comprise a flexible polymer section (such as a biocompatible polymer) that is rigidly or semi-rigidly fixed.

As mentioned briefly above, a stabilization device (including struts), may also include one or more coating or other surface treatment (embedding, etc.). Coatings may be protective coatings (e.g., of a biocompatible material such as a metal, plastic, ceramic, or the like), or they may be a bioactive coating (e.g., a drug, hormone, enzyme, or the like), or a combination thereof. For example, the stabilization device may elute a bioactive substance to promote or inhibit bone growth, vascularization, etc. In one variation, the device includes an elutible reservoir of bone morphogenic protein (BMP).

The stabilization device may be referred to as an inter (“between”) vertebral implant or inter-vertebral stabilization device. In some variations, multiple inter-vertebral stabilization device are used. For example, the devices may include bilateral devices (e.g., left and right). In some variations, a single, large device may be used. For example, the device typically extends between the adjacent vertebras in the expanded configuration; the device may also extend across a substantial lateral extent of the intervertebral space. The “bow” shape of the device may comprise an essentially flattened bow region, in which the struts are substantially flattened along their outer perimeter (e.g., having a box-like shape in the expanded configuration).

As previously mentioned, the stabilization device may be formed about a central elongate hollow body. In some variations, the struts are formed by cutting a plurality of slits long the length (distal to proximal) of the elongate body. This construction may provide one method of fabricating the stabilization device, however the stabilization device described herein are not limited to this construction. If formed in this fashion, the slits may be cut (e.g., by drilling, laser cutting, etc.) and the stabilization device may be pre-set or pre-biased into a deployed shape so that this configuration is the default, or relaxed, configuration in the body. For example, the struts may be formed by plastically deforming the material of the struts into the deployed configuration. In general, any of the stabilization devices may be thermally treated (e.g., annealed) so that they retain this deployed configuration when relaxed. Thermal treatment may be particularly helpful when forming a strut from a shape memory material such as a nickel-titanium alloy (e.g., Nitinol™) into the deployed configuration.

The stabilization devices described herein generally have two or more releasable inserter attachment regions for attaching to an inserter. For example, a stabilization device may include at a first attachment region for an inserter at the proximal end of the elongate shaft and a second attachment region for an inserter at the distal end of the elongate shaft. Generally the attachment regions for the inserter spans the stabilization device, so that the inserter may apply force to prevent the stabilization device from expanding until it has been inserted and/or positioned. For example, the inserter may apply force across the elongate shaft of the stabilization device (e.g., to apply tension to the struts and keep them in the contracted or delivery configuration). In some variations the stabilization device includes only a single attachment region (e.g., proximal to the stabilization device). In this variation, the device includes a seating region distal to the stabilization device against which a portion of an inserter (e.g., a rod) can press to apply force to control the configuration of the stabilization device. In some variations of the self-expanding stabilization device, the force to alter the configuration of the device from the delivery to the deployed configuration comes from the material of the elongate shaft itself (e.g., from a shape-memory material), and thus only a single attachment region (or one or more attachment region at a single end of the elongate shaft) is necessary.

Examples of inserters that may be used or adapted for use may be found in U.S. patent application Ser. No. 12/025,537, titled “Methods and Devices for Stabilizing Bone Compatible for use with Bone Screws”, filed Feb. 4, 2008, and U.S. patent application Ser. No. 12/024,938, titled “Systems, Devices and Methods for Stabilizing Bone”, filed Feb. 1, 2008, herein incorporated by reference.

### Inserter

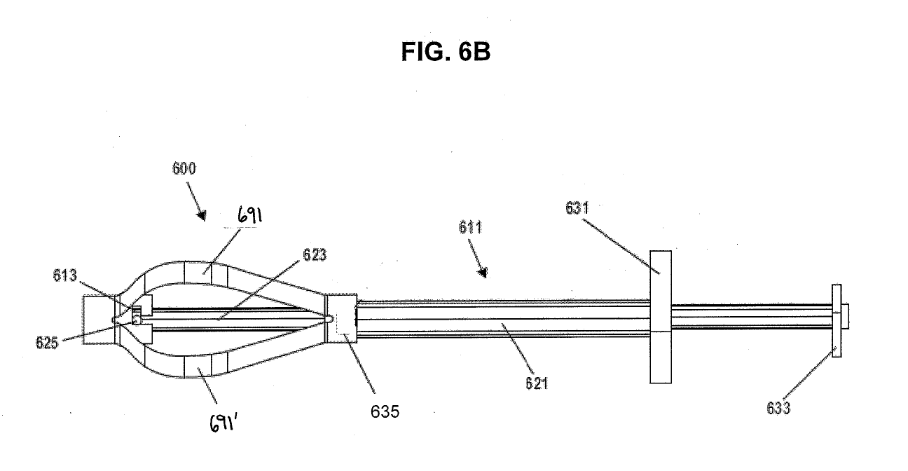

Returning now to FIG. 6A, an inserter may be used to insert an implant in a collapsed (delivery) configuration, and may be used to control the implantation of the device. For example, an inserter may initially attach to a stabilization implant at both the proximal and distal end of the implant. FIG. 6A shows a stabilization device 600 having a plurality of continuous curvature of bending struts 691, 691′ removably attached to an inserter 611. In this example, an attachment region 615 at the proximal portion of the stabilization device is configured as an L-shaped notch, as is the attachment region 613 at the distal portion of the device.

In general, an inserter includes an elongate body having a distal end to which the stabilization device may be attached and a proximal end which may include a handle or other manipulator that coordinates converting an attached stabilization device from a delivery and a deployed configuration, and also allows a user to selectively release the stabilization device from the distal end of the inserter.

The inserter 611 shown in FIG. 6A includes a first elongate member 621 that coaxially surrounds a second elongate member 623. In this variation, each elongate member 621, 623 includes a stabilization device attachment region at its distal end, to which the stabilization device is attached, as shown. In this example, the stabilization device attachment region includes a pin that mates with the L-shaped slots forming the releasable attachment regions on the stabilization device. In FIG. 6A the L-shaped releasable attachments on the stabilization device are oriented in opposite directions (e.g., the foot of each “L” points in opposite directions). Thus, the releasable attachment devices may be locked in position regardless of torque applied to the inserter, preventing the stabilization device from being accidentally disengaged.

The inserter shown in FIG. 6A also includes two grips 631, 633 at the proximal ends of each elongate member 621, 623. These grips can be used to move the elongate members (the first 621 or second 623 elongate member) relative to each other. The first and second elongate members of the inserter may be moved axially (e.g., may be slid along the long axis of the inserter) relative to each other, and/or they may be moved in rotation relative to each other (around the common longitudinal axis). Thus, when a stabilization device is attached to the distal end of the inserter, moving the first elongate member 621 axially with respect to the second elongate member 623 will cause the stabilization device to move between the deployed configuration (in which the struts are expanded) and the delivery configuration (in which the struts are relatively unexpanded). Furthermore, rotation of the first elongate member of the inserter relative to the second elongate member may also be used to disengage one or more releasable attachment regions of the stabilization device 613, 615 from the complementary attachment regions of the inserter 625, 627. Although the stabilization devices described herein are typically self-expanding stabilization devices, the inserter may be used with stabilization devices that do not self-expand. Even in self-expanding devices, the inserter may be used to apply additional force to convert the stabilization device between the delivery and the deployed configuration. For example, when allowed to expand in a cancellous bone, the force applied by the struts when self-expanding may not be sufficient to completely cut through the cancellous bone and/or distract the cortical bone as desired. In some variations, the inserter may also permit the application of force to the stabilization device to expand the struts even beyond the deployed configuration.

An inserter may also limit or guide the movement of the first and second elongate members, so as to further control the configuration and activation of the stabilization device. For example, the inserter may include a guide for limiting the motion of the first and second elongate members. A guide may be a track in either (or both) elongate member in which a region of the other elongate member may move. The inserter may also include one or more stops for limiting the motion of the first and second elongate members.

As mentioned above, the attachment regions on the inserter mate with the stabilization device attachments. Thus, the attachment regions of the inserter may be complementary attachments that are configured to mate with the stabilization device attachments. For example, a complimentary attachment on an inserter may be a pin, knob, or protrusion that mates with a slot, hole, indentation, or the like on the stabilization device. The complementary attachment (the attachment region) of the inserter may be retractable. For example, the inserter may include a button, slider, etc. to retract the complementary attachment so that it disconnects from the stabilization device attachment. A single control may be used to engage/disengage all of the complementary attachments on an inserter, or they may be controlled individually or in groups.

FIG. 6B is another variation of a stabilization device 600 releasably connected to an inserter 611, in which the attachment region 635 between the stabilization device and the inserter is configured as a screw or other engagement region, rather than the notch 615 shown in FIG. 6A.

In some variation the inserter includes a lock or locks that hold the stabilization device in a desired configuration. For example, the inserter may be locked so that the stabilization device is held in the delivery configuration (e.g., by applying force between the distal and proximal ends of the stabilization device). In an inserter such as the one shown in FIG. 6A, for example, a lock may secure the first elongate member to the second elongate member so that they may not move axially relative to each other.

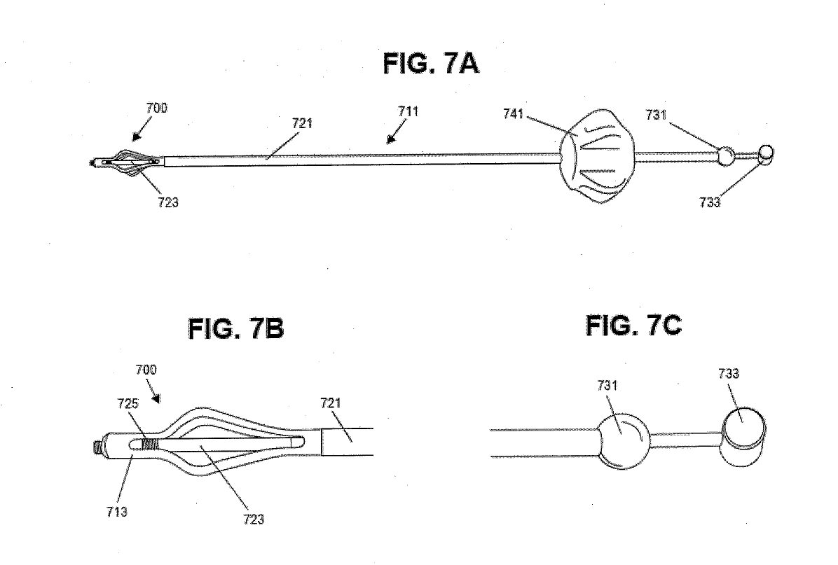

FIG. 7A is another example of an inserter 711 and an attached stabilization device 700. Similar to FIG. 6A, the stabilization device includes a first elongate member 721 attached to the proximal end of the stabilization device, and a second elongate member 723 attached to the distal end of the stabilization device. The first 721 and the second 723 elongate members are also configured coaxially (as a rod and shaft) that may be moved axially and rotationally independently of each other. The stabilization device 700 includes a plurality of continuous curvature of bending struts, shown in detail in FIG. 7B. The stabilization device 700 is shown in the deployed configuration. The distal end of the stabilization device includes a releasable attachment 713 that is configured as a threaded region which mates with a threaded complementary attachment 725 at the distal end of the structure.

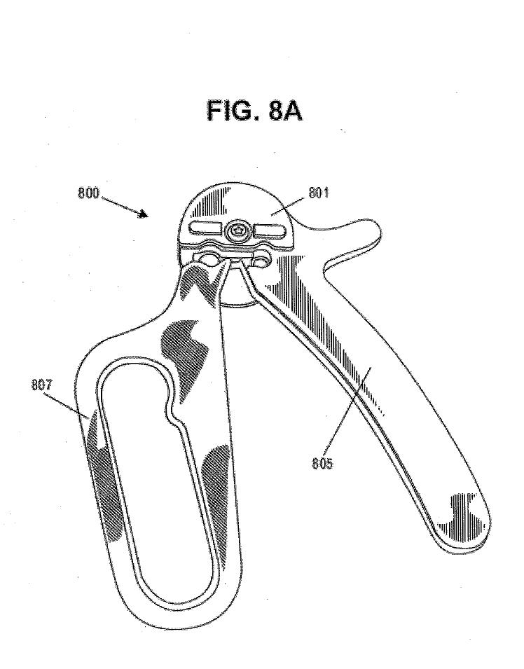

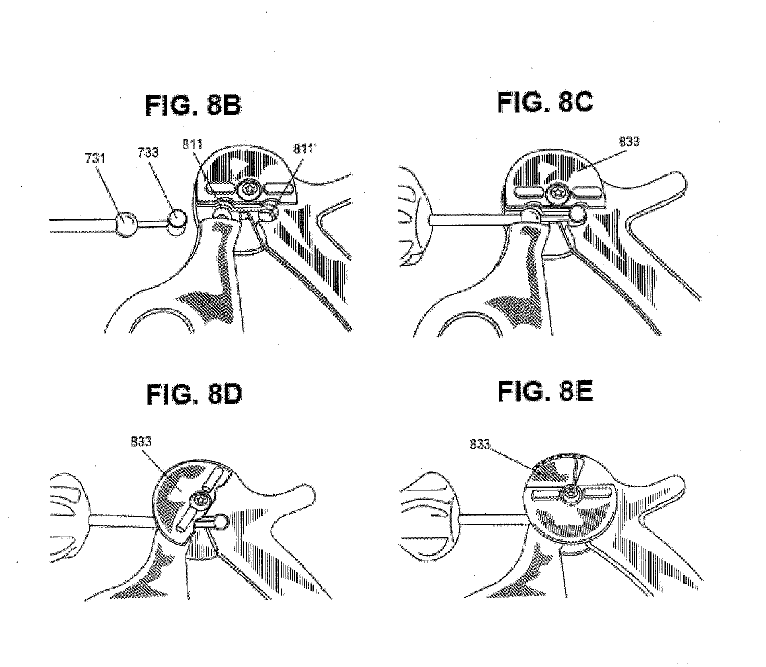

The proximal ends of the coaxial first and second elongated members 721, 723 also include grips 731, 733. These grips are shown in greater detail in FIG. 7C. As with the grips described in FIG. 6A, these grips may be grasped directly by a person (e.g., a physician, technician, etc.) using the device, or they may be connected to a handle. Thus, in some variations one or both grips are ‘keyed’ to fit into a handle, so that they can be manipulated by the handle. An example of this is shown in FIG. 8A-8E, and described below. The inserter of FIG. 7A also includes a knob 741 attached to the first elongated member 721 distal to the proximal end of the elongated member. This knob may also be used to move the first (or outer) elongate member of the inserter (e.g., to rotate it), or to otherwise hold it in a desired position. The knob may be shaped and/or sized so that it may be comfortably handheld.

Any of the inserters described herein may include, or may be used with, a handle. A handle may allow a user to control and manipulate an inserter. For example, a handle may conform to a subject's hand, and may include other controls, such as triggers or the like. Thus, a handle may be used to control the relative motion of the first and second elongate members of the inserter, or to release the connection between the stabilization device and the inserter, or any of the other features of the inserter described herein.

An inserter may be packaged or otherwise provided with a stabilization device attached. Thus, the inserter and stabilization device may be packaged sterile, or may be sterilizable. In some variations, a reusable handle is provided that may be used with a pre-packaged inserter stabilization device assembly. In some variations the handle is single-use or disposable. The handle may be made of any appropriate material. For example, the handle may be made of a polymer such as polycarbonate.

FIG. 8A illustrates one variation of a handle 800 that may be used with an inserter, such as the inserter shown in FIGS. 7A-7C. The handle 800 includes a hinged joint 803, and the palm contacting 805 region and finger contacting 807 region of the handle 800 may be moved relative to each other by rotating about this hinged joint 803. This variation of a handle also includes a thumb rest 809, which may also provide additional control when manipulating an inserter with the handle. The thumb rest may also include a button, trigger, or the like.

FIGS. 8B-8E illustrate the connection of an inserter such as the inserter described above in FIGS. 7A-C into a handle 800. In FIG. 8B the proximal end of the inserter is aligned with openings 811, 811′ in the handle. These openings are configures so that the grips 731, 733 at the distal ends of the first and second elongate members of the inserter can fit into them. In this example, the grip 733 is shaped so that it can be held in the opening 811′ of the handle in an oriented fashion, preventing undesirable rotation. Thus, in FIG. 8C the proximal end of the inserter (the grips 731 and 732) are placed in the openings 811, 811′. The inserter may then be secured to the handle by rotating cover 833, as shown in FIGS. 8D and 8E.

By securing the proximal end of the inserter in the handle, the handle can then be used to controllably actuate the inserter, as illustrated in FIGS. 9A-9D. In this example the stabilization device is in the deployed configuration (shown in FIG. 9A) when the handle is “open” (shown in FIG. 9B). By squeezing the handle (rotating the finger grip region towards the palm region, as shown in FIG. 9D) the inserter applies force between the proximal and distal regions of the stabilization device, placing it in a delivery configuration, as shown in FIG. 9C.

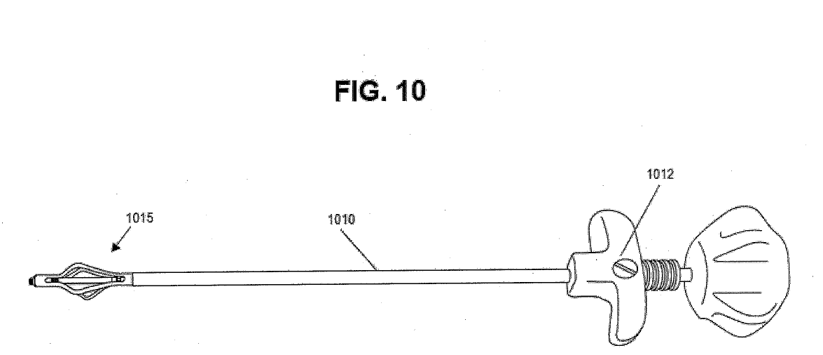

As mentioned above, in the delivery configuration the struts of the stabilization device are typically closer to the long axis of the body of the stabilization device. Thus, the device may be inserted into the body for delivery into a bone region. This may be accomplished with the help of an access cannula (which may also be referred to as an introducer). As shown in FIG. 10, the inserter 1015 is typically longer than the access cannula 1010, allowing the stabilization device to project from the distal end of the access cannula for deployment. The access cannula may also include a handle 1012.

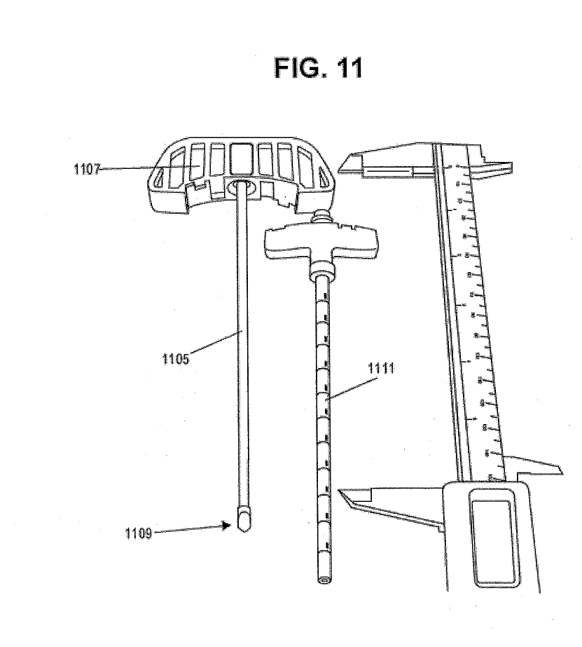

Any of the devices (stabilization devices) and inserters (including handles) may be included as part of a system or kit for correcting a bone defect or injury. FIGS. 10 through 14D illustrate different examples of tools (or variations of tools) that may be used as part of a system for repair bone. Any of these tools (or additional tools) may also be used to perform the methods of repairing bone (particularly spinal bone) described herein. For example, FIG. 11 shows a trocar 1105 having a handle 1107 and a cutting/obdurating tip 1109. This trocar 1105 may also be used with an access cannula 1111. Another example of an access cannula 1111 (or introducer) is shown adjacent to the trocar 1106 in FIG. 11. This exemplary access cannula has an inner diameter of approximately 4.2 mm, so that the trocar 1105 will fit snugly within it, and a stabilization device in a delivery configuration will also fit therein. Any appropriate length cannula and trocar may be used, so long as it is correctly scaled for use with the introducer and stabilization device. For example, the access cannula may be approximately 15.5 cm long. The trocar an introducer may be used to cut through tissue until reaching bone, so that the introducer can be positioned appropriately.

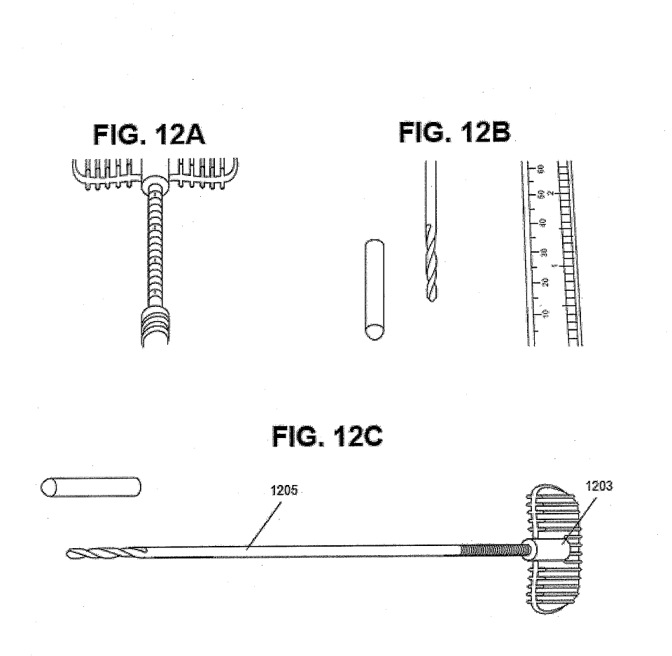

A bone drill, such as the hand drill shown in FIGS. 12A-12C, may then be used to access the cancellous bone. The twist drill 1201 shown in FIG. 12A-12C has a handle 1203 at the proximal end and a drill tip 1205 at the distal end. This twist drill may be used with the same access cannula previously described (e.g., in this example the twist drill has an outer diameter of 4.1 mm and a length of 19.5 cm). The distal (drill) end of the twist drill may extend from the cannula, and be used to drill into the bone. The proximal end of the twist drill shown in FIGS. 12A-12C is calibrated (or graduated) to help determine the distance drilled.

Any of the devices shown and described herein may also be used with a bone cement. For example, a bone cement may be applied after inserting the stabilization device into the bone, positioning and expanding the device (or allowing it to expand and distract the bone) and removing the inserter, leaving the device within the bone. Bone cement may be used to provide long-term support for the repaired bone region.

Any appropriate bone cement or filler may be used, including PMMA, bone filler or allograft material. Suitable bone filler material include bone material derived from demineralized allogenic or xenogenic bone, and can contain additional substances, including active substance such as bone morphogenic protein (which induce bone regeneration at a defect site). Thus materials suitable for use as synthetic, non-biologic or biologic material may be used in conjunction with the devices described herein, and may be part of a system includes these devices. For example, polymers, cement (including cements which comprise in their main phase of microcrystalline magnesium ammonium phosphate, biologically degradable cement, calcium phosphate cements, and any material that is suitable for application in tooth cements) may be used as bone replacement, as bone filler, as bone cement or as bone adhesive with these devices or systems. Also included are calcium phosphate cements based on hydroxylapatite (HA) and calcium phosphate cements based on deficient calcium hydroxylapatites (CDHA, calcium deficient hydroxylapatites). See, e.g., U.S. Pat. No. 5,405,390 to O'Leary et al.; U.S. Pat. No. 5,314,476 to Prewett et al.; U.S. Pat. No. 5,284,655 to Bogdansky et al.; U.S. Pat. No. 5,510,396 to Prewett et al.; U.S. Pat. No. 4,394,370 to Jeffries; and U.S. Pat. No. 4,472,840 to Jeffries, which describe compositions containing demineralized bone powder. See also U.S. Pat. No. 6,340,477 to Anderson which describes a bone matrix composition. Each of these references is herein incorporated in their entirely.

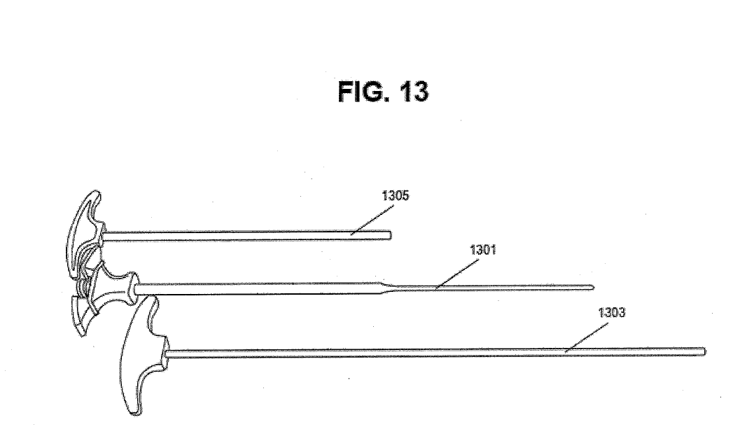

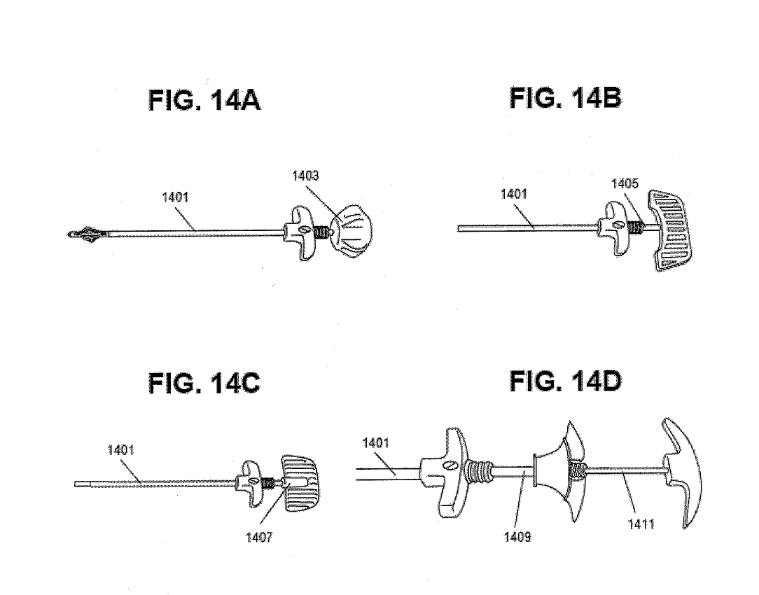

FIG. 13 shows a tapered cement cannula 1301 that may be used to deliver bone cement to the insertion site of the device, and also shows two cement obturators 1303, 1305 for delivering the cement (piston-like). The cannula delivering cement is also designed to be used through the access cannula, as are all of the components described above, including the stabilization device and inserter, trocar, and drill. This is summarized in FIGS. 14A-14D. FIG. 14A illustrates an access cannula 4101 with a stabilization device 1403 and inserter inserted through the access cannula, as shown in FIG. 10. FIG. 14B shows a trocar 1405 within the access cannula 1401. FIG. 14C shows a hand drill 1407 within the same access cannula 1401, and FIG. 14D shows a cement cannula 1409 and a cement obturator 1411 within the same access cannula 1401. These devices may be used to repair a bone, or to fix or fuse bone, including vertebra. These devices may be used for insertion and implantation into bone (e.g., vertebra) and even softer tissue, particularly vertebral disc tissue, as described in greater detail below, following an illustration of the use of the device within a vertebra.

### Exemplary Method of Repairing a Bone

As mentioned above, any of the devices described herein may be used to repair a bone or disk. Implantation of the stabilization device may be performed as illustrated below for treatment of a vertebra by delivering a stabilization device (e.g., a self-expanding stabilization device as described herein) within a cancellous bone region, and allowing the device to expand within the cancellous bone region so that a cutting surface of the device cuts through the cancellous bone. This example illustrates the general operation and variations of the devices and methods described, and may also be applied to the PLIF methods, including the method of insertion into a disc and fusion of multiple vertebra, as discussed further below.

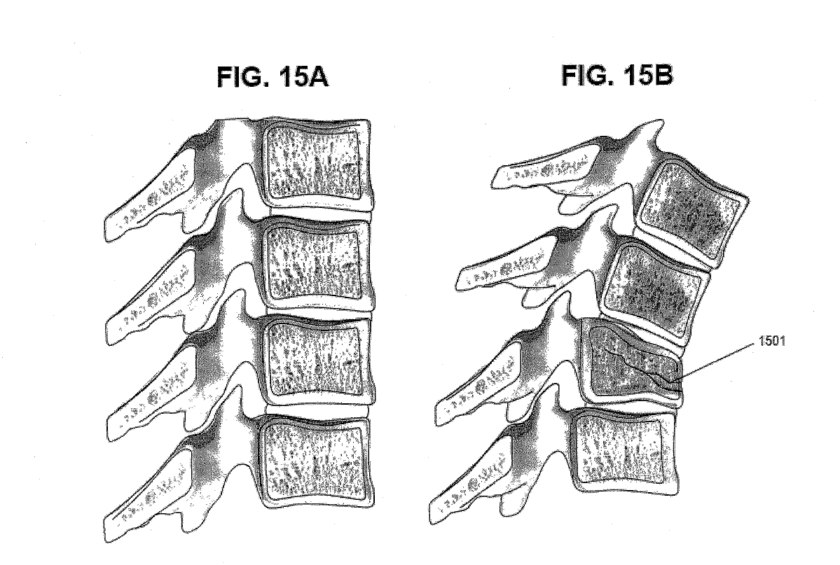

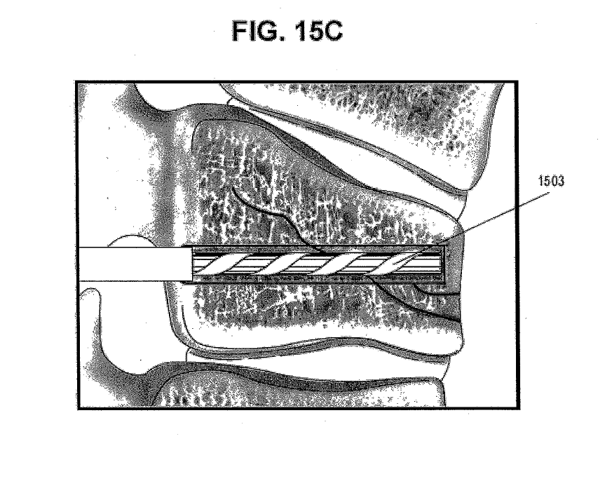

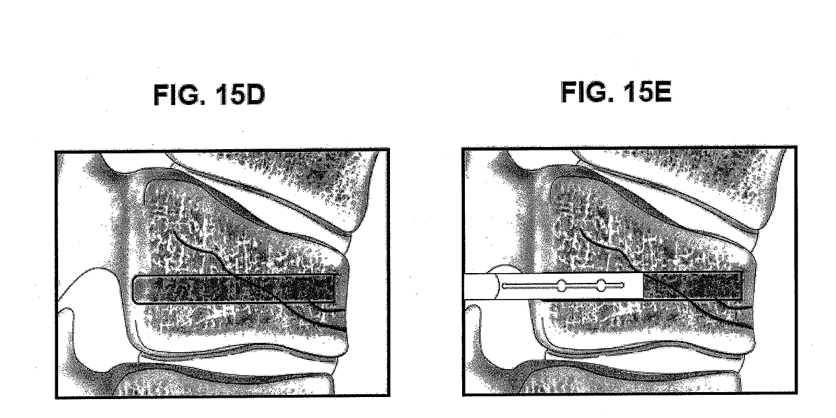

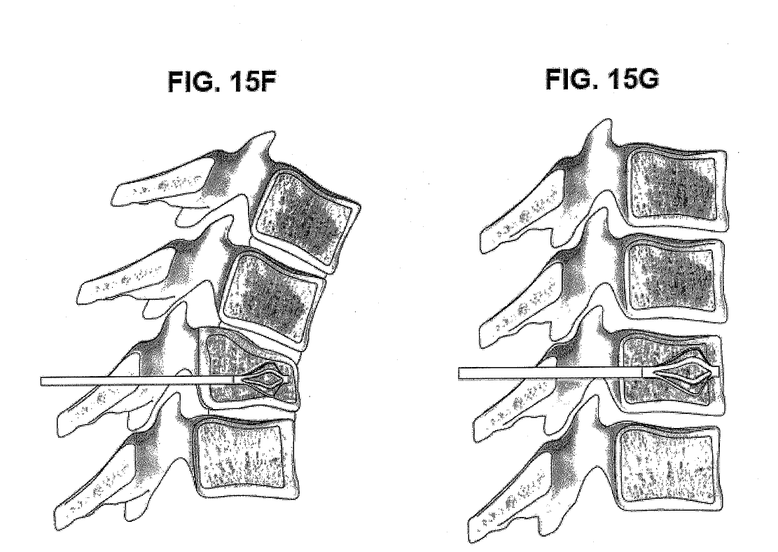

In addition to fusion of vertebra, a stabilization device as described herein may be used to repair a compression fracture in spinal bone (either with or without additionally fusing the vertebra). This is illustrated schematically in FIGS. 15A-15G. FIG. 15A shows a normal thoracic region of the spine in cross-section along the sagital plane. The spinal vertebra are aligned, distributing pressure across each vertebra. FIG. 15B shows a similar cross-section through the spine in which there is a compression fracture in the 11^{th} thoracic vertebra 1501. The 11^{th} vertebra is compressed in the fractured region. It would be beneficial to restore the fractured vertebra to its uninjured position, by expanding (also referred to as distracting) the vertebra so that the shape of the cortical bone is restored. This may be achieved by inserting and expanding one of the stabilization devices described herein. In order to insert the stabilization device, the damaged region of bone must be accessed.

As mentioned above, an introducer (or access cannula) and a trocar, such as those shown in FIG. 11 may be used to insert the access cannula adjacent to the damaged bone region. Any of the steps described herein may be aided by the use of an appropriate visualization technique. For example, a fluoroscope may be used to help visualize the damaged bone region, and to track the p of inserting the access cannula, trocar, and other tools. Once the access cannula is near the damaged bone region, a bone drill may be used to drill into the bone, as shown in FIG. 15C.

In FIG. 15C the drill 1503 enters the bone (or in some variations, the disc) from the access cannula. The drill enters the cancellous bony region within the vertebra. After drilling into the vertebra to provide access, the drill is removed from the bone and the access cannula is used to provide access to the damaged vertebra, as shown, by leaving the access cannula in place, providing a space into which the stabilization device may be inserted in the bone, as shown in FIG. 15D. In FIG. 15E a stabilization device, attached to an inserter and held in the delivery configuration, is inserted into the damaged vertebra.

Once in position within the vertebra, the stabilization device is allowed to expand (by self-expansion) within the cancellous bone of the vertebra, as shown in FIG. 15F. In some variations, the device may fully expand, cutting through the cancellous bone and pushing against the cortical bone with a sufficient restoring force to correct the compression, as shown in FIG. 15G. However, in some variations, the force generated by the device during self-expansion is not sufficient to distract the bone, and the inserter handle may be used (e.g., by applying force to the handle, or by directly applying force to the proximal end of the inserter) to expand the stabilization device until the cortical bone is sufficiently distracted.

Once the stabilization device has been positioned and is expanded, it may be released from the inserter. In some variations, it may be desirable to move or redeploy the stabilization device, or to replace it with a larger or smaller device. If the device has been separated from the inserter (e.g., by detaching the removable attachments on the stabilization device from the cooperating attachments on the inserter), then it may be reattached to the inserter. Thus, the distal end of the inserter can be coupled to the stabilization device after implantation. The inserter can then be used to collapse the stabilization device back down to the delivery configuration (e.g., by compressing the handle in the variation shown in FIGS. 9A-9D), and the device can be withdrawn or re-positioned.

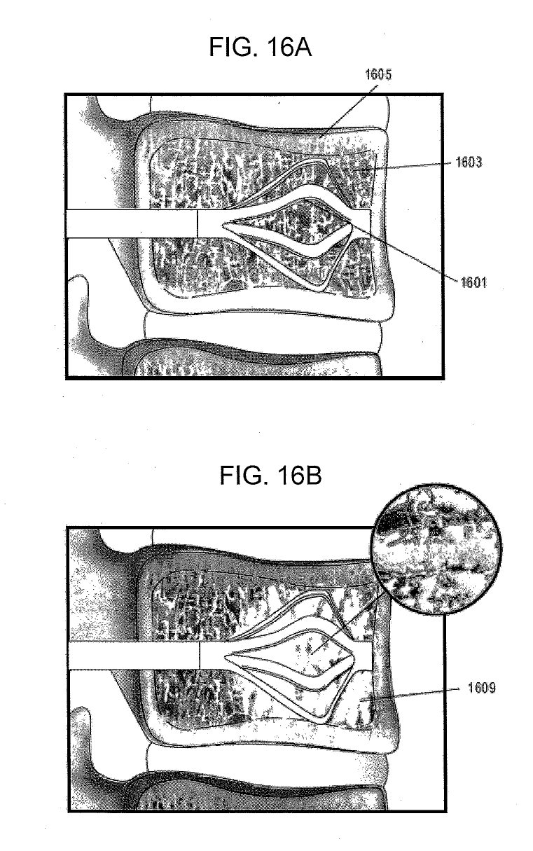

As mentioned above, a cement or additional supporting material may also be used to help secure the stabilization device in position and repair the bone. For example, bone cement may be used to cement a stabilization device in position. FIGS. 16A-16C illustrate one variation of this. In FIG. 16A the stabilization device 1601 has been expanded within the cancellous bone 1603 and is abutting the cortical bone 1605. Although in some variations the addition of the stabilization device may be sufficient to repair the bone, it may also be desirable to add a cement, or filler to help secure the repair. This may also help secure the device in position, and may help close the surgical site.

For example, in FIG. 16B a fluent bone cement 1609 has been added to the cancellous bone region around implant. This cement will flow through the channels of trebeculated (cancellous) bone, and secure the implant in position. This is shown in greater detail in the enlarged region. This bone cement or filler can be applied using the delivery cannula (e.g., through a cement cannula, as described above), and allowed to set.

Methods of performing a Posterior Lumbar Interbody Fusion (PLIF) Surgery

As mentioned above, any of the stabilization devices described herein may be used in a PLIF surgery. A method of performing a posterior lumbar interbody fusion surgery described herein typically involves accessing a posterior portion of an intervertebral disc, forming a channel in the intervertebral disc, inserting a stabilization device in the channel or opening, expanding (e.g., self-expanding) the device in the channel until the struts of the stabilization device cut through the intervertebral disc and contact both vertebrae adjacent to the intervertebral disc, and injecting the space around the stabilization device with a flowable material such as a bone graft or other material to promote fusion of the adjacent vertebrae. The order of these steps may be different or some of the steps may be combined. For example, the stabilization device may be driven into the intervertebral disc without first forming a separate channel. Thus, the stabilization device itself may be used to form the channel by insertion into the intervertebral disc. Other additional steps are described below.

The general step of accessing a posterior portion of an intervertebral disc may include the step of making an incision in a patient. The length of the incision may be as small as 1 cm, for example, which would be small enough to admit an inserter containing one embodiment of a stabilization device in the delivery configuration. The step of accessing a posterior portion of the intervertebral disc may also include stripping or retracting the paraspinous tissues away from the spine. The accessing step may further include performing a bone removal procedure, such as a laminectomy, a lumbar discectomy, or trimming the facet joints to allow visualization of the spinal nerve roots and the intervertebral disc.

The general step of forming a channel in the intervertebral disc may include the step of drilling a channel in the intervertebral disc. The drilled hole or cavity is typically large enough to fit at least a portion of the length of the stabilization device into the intervertebral disc. Alternatively, the channel can be formed by hammering, chiseling, or reaming out a channel in the intervertebral disc. In an alternative embodiment, the stabilization device can be fitted with a sharp or pointy distal end, and can be driven directly into the intervertebral disc to form a channel, without first having to form a channel before inserting the stabilization device. The width is typically large enough to accommodate the width of the distal end of the stabilization device in the collapsed or delivery configuration; the width is typically smaller than the expanded diameter of the stabilization device. In some variations the stabilization device is configured to expand between about 1.5 to 5 times the diameter of the collapsed (delivery) configuration. The intervertebral disc opening into which the device is inserted may also be a naturally occurring or pre-existing disc opening, depending upon the intended application. In an alternate method of performing a PLIF surgery, the general step of forming a channel in a first and second vertebra is similar to the step of forming a channel in an intervertebral disc, as described above.

When inserting the stabilization device within the intervertebral disc (or, alternatively, inserting the stabilization device within the vertebrae adjacent to the intervertebral disc), the stabilization device is typically held in the contracted position using an inserter, which may then be used to position the device in the spine. Once the position is achieved, the stabilization device may be released (all at once, or gradually) from the inserter so that it can self-expand within the channel. The struts may therefore bow outward and contact the walls of the cavity.

Depending on the stabilization device, the area of the spine, the sizes of the device and the channels, the struts may cut through the vertebrae or intervertebral disc (at least partially), thereby further anchoring it. After allowing the stabilization device to self-expand, additional force may optionally be applied to further expand the device. For example, the inserter may apply compressive force over the stabilization device to further expand it. When the stabilization device is inserted into an intervertebral disc, further force may need to be applied to fully expand the stabilization device so that it contacts both vertebrae adjacent to the intervertebral disc. The inserter may be removed from the stabilization device. For example, the attachment sites between the inserter and the stabilization device (typically at either end of the stabilization device) may be disengaged by unscrewing or unlocking, e.g., by rotating, by activating a push-button, etc. In some variations the stabilization device may be repositioned by re-engaging the inserter with the stabilization device and applying tension across the stabilization device to collapse it again so that it can be repositioned.

Once the stabilization device is positioned in the bone, it may be further secured in place by the injecting a flowable material (e.g., bone cement, bone graft, or other fluent material) or securing screw, pin, etc. The flowable material is also used to promote fusion of the vertebrae adjacent to the intervertebral disc. If a bone cement is used, any appropriate cement may be used, including flowable materials, such as a bone filling composition or cement, which may include biological materials, synthetic materials, inorganic materials, or bioactive agents (or any combinations thereof). Other bone filling compositions or cement include PMMA (polymethylmethacrylate) which may be injected into the cavity into which the stabilization device has been positioned. In particular, the central passageway through the stabilization device may be used to deliver the flowable material. For example, material may be delivered through a trocar and cannula into the passageway of a device.

There are many suitable materials known in the art for filling in vacant spaces in bone and which may be used herein. Some of these materials or compositions are biological in origin and some are synthetic, as described in U.S. patent application Ser. No. 11/468,759, which is incorporated by reference herein. The material may be applied to flow into the open space within the stabilization device and to some degree, into the peripheral area surrounding the device. The device may be capped or blocked to prevent excess loss of material applied, and help confine it somewhat to the spine. The process of applying flowable material may also be observed to control the amount and location applied. For example, a flowable cementing material may contain radiopaque material so that when injected under live fluoroscopy, cement localization and leakage can be observed.

Another example of bone cementing material is a ceramic composition including calcium sulfate calcium hydroxyapatite, such as Cerament™, as manufactured by BoneSupport AB (Lund, Sweden). Ceramic compositions provide a dynamic space for bone in-growth in that over time, they resorb or partially resorb, and as a consequence provide space for in-growth of new bone. Bioactive agents may also be included in a cementing composition, such as osteogenic or osteoinductive peptides, as well as hormones such at parathyroid hormone (PTH). Bone Morphogenetic Proteins (BMPs) are a prominent example of effective osteoinductive agents, and accordingly, a protein such as recombinant human BMP-2 (rhBMP-2) may included in an injected bone-filling composition. In this particular context, BMPs promote growth of new bone into the regions in the interior of the expanded struts and around the periphery of the device in general, to stabilize the device within new bone. A more fundamental benefit provided by the new bone growth, aside from the anchoring of the device, is simply the development of new bone which itself promotes healing. In some variations, antibiotics may be included. In general, any appropriate flowable material may be injected into the device or the channel surrounding the device. Some variations of the devices described herein include a passageway for a flowable material through the entire length of the device.

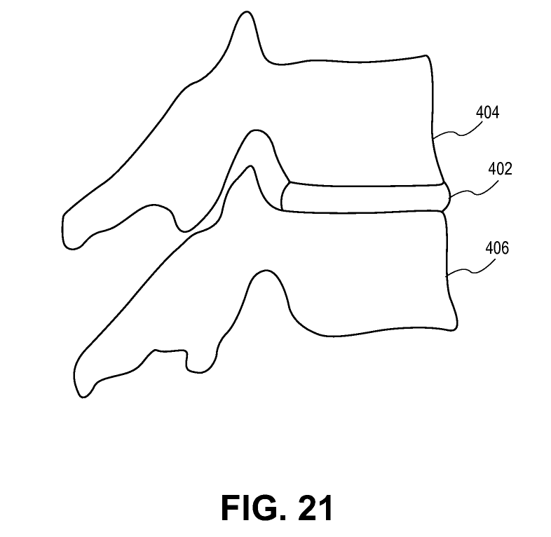

FIG. 4 illustrates a portion of a human spine, including an intervertebral disc 402, a first vertebra 404, and a second vertebra 406. The posterior portion of the spine is shown on the left side of FIG. 21, and the anterior portion of the spine is shown on the right side of FIG. 21. Other features of a human spine can be seen in FIG. 21, including a spinous process and a superior articular process, as known in the art.

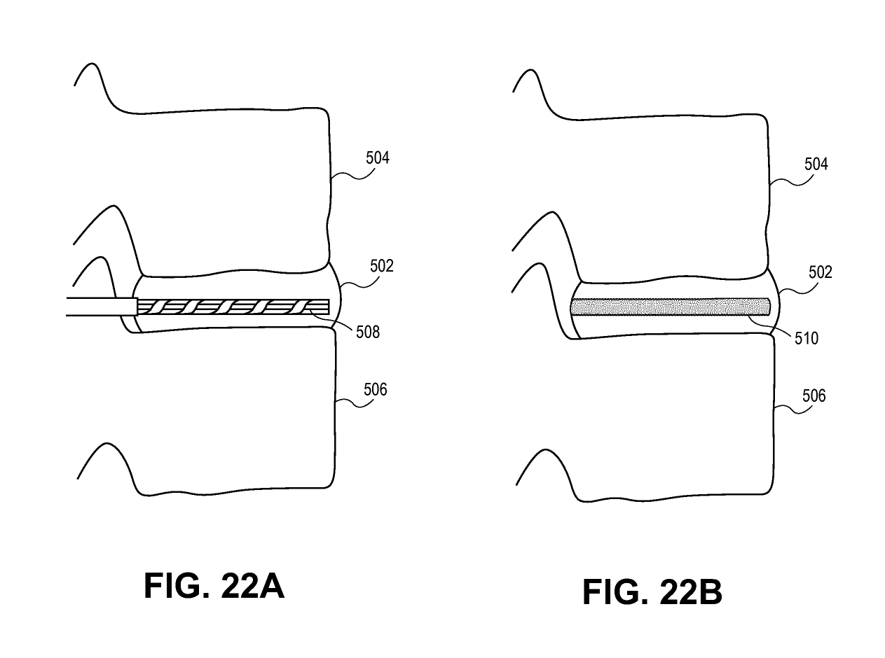

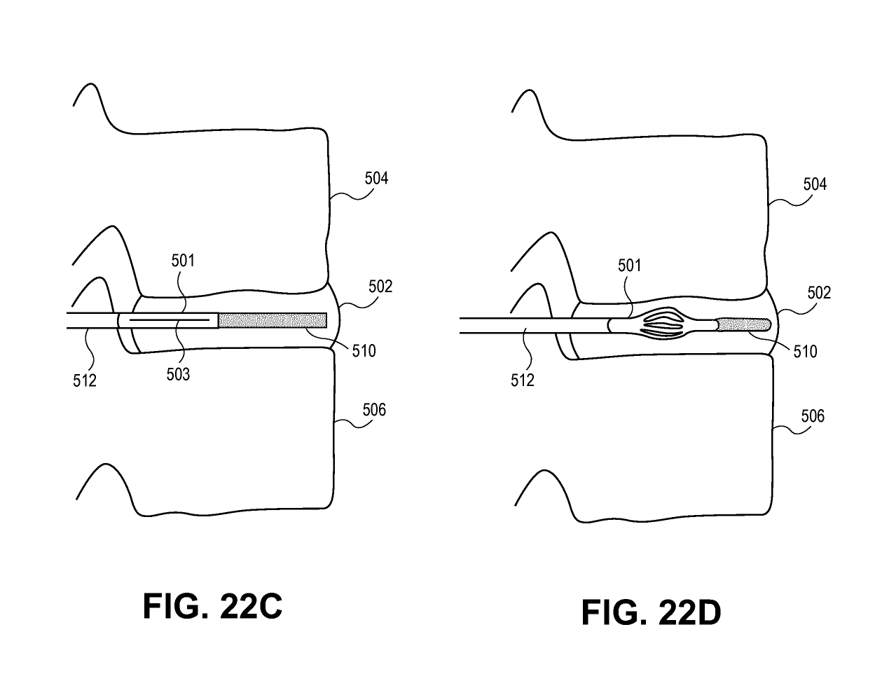

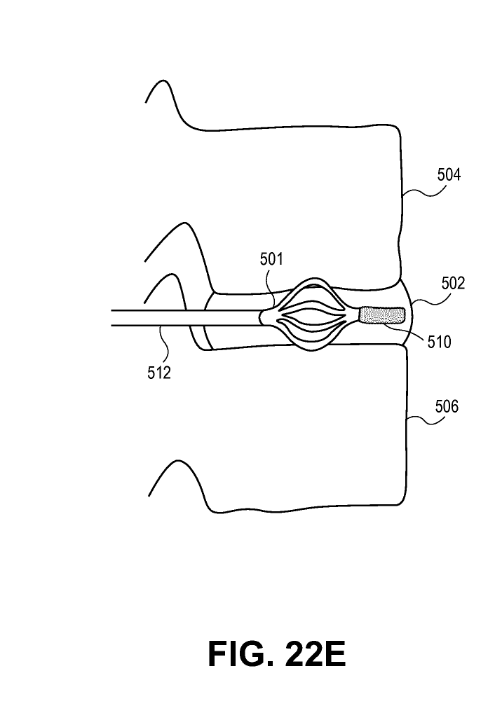

FIGS. 22A-22E illustrate one variation of a method of performing a PLIF surgery using a system and device as described herein. In this example, a stabilization device is used in a PLIF surgery. FIG. 22A illustrates forming a channel or passageway within the intervertebral disc 502 with drill 508. An elongate instrument is used to drill the hole, and the width and depth may be matched to the stabilization device to be used, or the stabilization device can be matched to the size of the channel formed. The passageway can be formed by any appropriate method and is not limited to drilling. For example, a channel may be found or enlarged by hammering, chiseling, reaming, etc. As shown in FIG. 22A, drill 508 accesses a posterior portion of intervertebral disc 502. As described above, this can be achieved by making an incision in a patient's back and inserting the drill into the incision, for example.

FIG. 22B shows channel 510 prepared to receive a stabilization device. FIG. 22C shows the distal end of one variation of an inserter/delivery device 512 within the formed channel 510. In this example the inserter is a cannula device having an outer sleeve into which the stabilization device 501 is held. The cannula applies radial force to keep the stabilization device compressed. A push rod in the cannula may be used to eject the stabilization device. In some variations this push rod is releasably connected to the stabilization device, which may allow the position of the device to be adjusted (e.g., by pulling the device back into the cannula).

In FIG. 22C the stabilization device 501 is just beginning to exit the cannula sleeve that radially envelopes the stabilization device 501 being inserted. FIG. 22D shows the stabilization device 501 after partial expansion of the self-expanding struts once the majority of the elongate shaft of the stabilization device 501 has been pushed out of the distal end of the cannula 512. The cannula has also been partially withdrawn from the implant site as the device is inserted. In some variations, the device may fully expand, cutting through the intervertebral disc 502 and pushing against both first vertebra 504 and second vertebra 506. However, in some variations, the force generated by the device during self-expansion is not sufficient to cut through the intervertebral disc, and force may be applied (e.g., by applying force to the inserter) to further expand the stabilization device within the intervertebral disc. FIG. 22E shows the stabilization device 501 fully expanded within the intervertebral disc so the struts of the stabilization device contact first vertebra 504 and second vertebra 506.

A bone cement or other flowable material may also be applied or injected to fill the region around the stabilization device shown in FIG. 22E to promote fusion of the adjacent vertebrae. For example, the cannula of the inserter may be used to apply a flowable material into the central passageway of the stabilization device, as previously described. A bone cement or filler may further help secure the stabilization device in the intervertebral disc and promote fusion of first vertebra 504 to second vertebra 506.

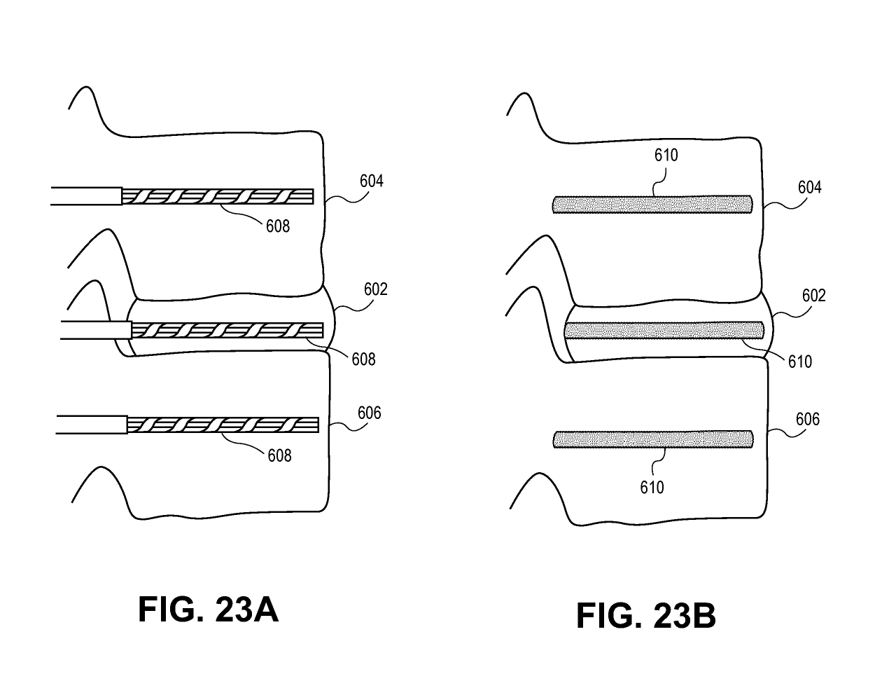

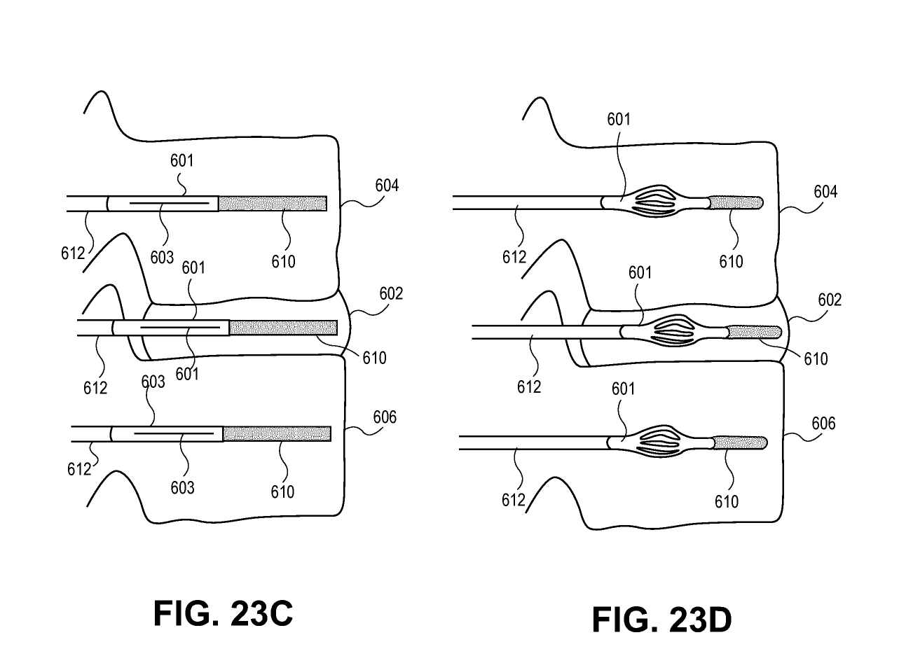

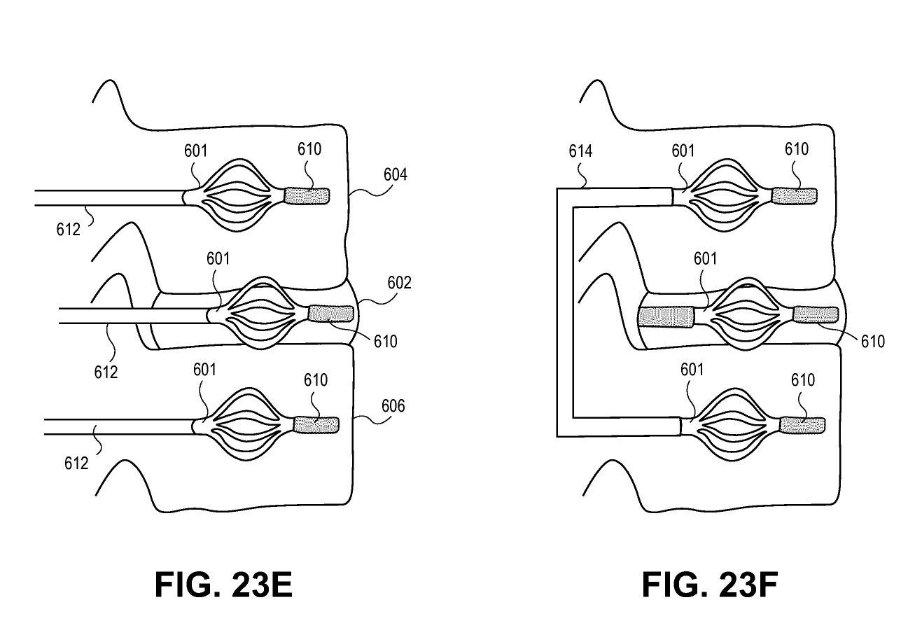

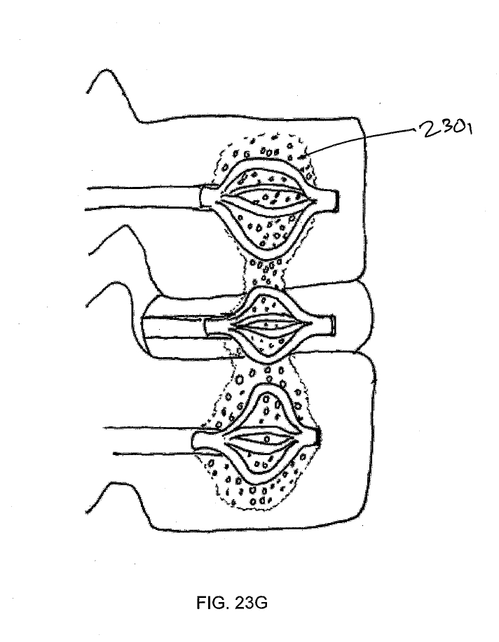

FIGS. 23A-6F illustrate one variation of a method of performing a PLIF surgery using a system and device as described herein. The method steps illustrated by FIGS. 23A-6E are similar to the steps described above and illustrated in FIGS. 22A-5E, but include additional stabilization devices in the vertebrae adjacent to the intervertebral disc to provide additional support and bracing and aid in fusion.

Thus, FIG. 23A illustrates forming a first channel within intervertebral disc 602, a second channel within first vertebra 604, and a third channel within second vertebra 606. As described above, the channels can be formed with drill 608, or by other methods such as hammering, chiseling, or reaming, for example. The channels formed in the first and second vertebrae may be larger than the channel formed in the intervertebral disc, since the vertebrae are larger and can accommodate a larger stabilization device for better anchoring and stability. However, the same size channels may be formed in both vertebrae and the intervertebral disc. As shown in FIG. 23A, drill 608 accesses the posterior portion of the spine, as described above. For ease of illustration, drill 608 is shown in each of the intervertebral disc and first and second vertebrae, however it should be understood that each of the channels need not be formed simultaneously but can be formed one at a time.

FIG. 23B shows channels 610 in the intervertebral disc and the first and second vertebrae, each channel being prepared to receive a stabilization device. FIG. 23C shows stabilization devices 601 beginning to exit the cannula sleeve as they are being inserted into channels 610. FIG. 23D shows the stabilization devices 601 after partial expansion of the self-expanding struts once the majority of the elongate shaft of the stabilization devices 601 have been pushed out of the distal end of the cannula 612. In some variations, the devices may fully expand, cutting through the intervertebral disc 602, first vertebra 604, or second vertebra 606. However, in some variations, the force generated by the devices during self-expansion is not sufficient to cut through the intervertebral disc or vertebrae, and force may be applied (e.g., by applying force to the inserter) to further expand the stabilization devices within the channels. FIG. 23E shows the stabilization devices 601 fully expanded within the intervertebral disc and vertebra. The stabilization device located within the intervertebral disc is expanded until the struts contact both the first vertebra 604 and the second vertebra 606.

As described above, a bone cement or other flowable material may also be applied or injected to fill the region around the stabilization devices shown in FIG. 23E to promote fusion of the adjacent vertebrae. For example, the cannula of the inserter may be used to apply a flowable material into the central passageway of the stabilization device, as previously described. A bone cement or filler may further help secure the stabilization device in the intervertebral disc and promote fusion of first vertebra 604 to second vertebra 606.

FIG. 23F shows connecting member 614 which is adapted to connect the stabilization devices implanted within the first and second vertebrae. Any appropriate connecting member can be used to connect the stabilization devices. For example, a connector (also referred to as a connecting member or a connector region) may include pins, rods, screws, bendable or flexible materials, bio-absorbable materials, strings, wires, or other methods as known in the art. Connecting the stabilization devices within the adjacent vertebrae provides another level of support and bracing which can hold the vertebrae together to prevent them from moving and promote fusion.

In some variations, the stabilization implants may be tied together (e.g., fused) by the use of the flowable material, and particularly settable flowable materials, such as cements (e.g., PMMA). For example, FIG. 23G shows an alternate variation of FIG. 23G in which the implants inserted into two adjacent vertebra and the disc, have all been at least partially fused together by the use of a cement 2301 through the devices. The flowable material in any of these variations may be inserted through and around the device and allowed to at least partially surround (e.g., flow into) the tissue, or it may be contained within a bag or other structure either within or around the implants.

The addition of material including the flowable material around the implants as shown in FIG. 23G and FIG. 16B may greatly enhance the strength of the implant and therefore (during PLIF) the fusion.

While the devices, systems, and methods for using them have been described in some detail here by way of illustration and example, such illustration and example is for purposes of clarity of understanding only. It will be readily apparent to those of ordinary skill in the art in light of the teachings herein that certain changes and modifications may be made thereto without departing from the spirit and scope of the invention.

We claim:

1. A method of fusing adjacent vertebrae comprising:
accessing a posterior portion of an intervertebral disc;
forming a channel in the intervertebral disc;
inserting an elongate stabilization device into the channel, the stabilization device having a plurality of self-expanding struts that are held in a collapsed configuration during insertion by applying force to push apart the proximal and distal ends of the stabilization device;
expanding the stabilization device until the struts contact first and second vertebrae adjacent to the intervertebral disc; and
injecting a flowable material around the stabilization device to promote fusion of the first and second vertebrae.

2. The method of claim 1, wherein the step of expanding comprises reducing or releasing the force applied to push apart the proximal and distal ends of the stabilization device.

3. The method of claim 1, wherein the step of expanding comprises holding the distal end of the stabilization device substantially fixed and allowing the proximal end of the stabilization device to foreshorten during expansion.

4. The method of claim 1 further comprising applying force to further expand the stabilization device within the intervertebral disc.

5. The method of claim 1, wherein the stabilization device cuts through the intervertebral disc during the expanding step.

6. The method of claim 5, wherein at least some of the self-expanding struts include a cutting surface adapted to cut through the intervertebral disc.

7. The method of claim 1, wherein the accessing step comprises making an incision into a patient.

8. The method of claim 7, wherein the incision is less than 1 cm in length.

9. The method of claim 1 further comprising visualizing the stabilization device within the intervertebral disc.

10. The device of claim 1, wherein the stabilization device is formed of a shape memory material.

11. The method of claim 1, wherein the flowable material is a biologic or synthetic material to promote anchoring and to allow for new bone in growth.

12. A method of fusing adjacent vertebrae comprising:
accessing a posterior portion of a spine;
forming a first channel in an intervertebral disc, a second channel in a first vertebra adjacent to the intervertebral disc, and a third channel in a second vertebra adjacent to the intervertebral disc;
inserting a first stabilization device into the first channel, inserting a second stabilization device into the second channel, inserting a third stabilization device into the third channel, each of the stabilization devices having a plurality of self-expanding struts extendable therefrom, the struts held in an insertion configuration by applying force to push apart the proximal and distal ends of each stabilization device;
expanding the stabilization devices, wherein the first stabilization device is expanded until the struts contact the first and second vertebrae;
injecting a flowable material around the stabilization devices; and
attaching the second stabilization device to the third stabilization device.

13. The method of claim 12 further comprising applying force to further expand the stabilization device within the intervertebral disc.

14. The method of claim 12, wherein the stabilization device cuts through the intervertebral disc during the expanding step.

15. The method of claim 12, wherein at least some of the self-expanding struts of the stabilization devices include a cutting surface adapted to cut through the intervertebral disc.

16. The method of claim 12, wherein the accessing step comprises making an incision into a patient.

17. The method of claim 16, wherein the incision is less than 1 cm in length.

18. The method of claim 12 further comprising visualizing the device during the inserting and/or expanding steps.

19. The device of claim 12, wherein the stabilization device is formed of a shape memory material.

20. The method of claim 12, wherein the flowable material is a biologic or synthetic material to promote anchoring and to allow for new bone in growth.
